(19) United States
(12) Patent Application Publication
(10) Pub. No.: US 2025/0186607 A1
(43) Pub. Date: Jun. 12, 2025

(54) ONE-STEP SUPRAMOLECULAR MULTIFUNCTIONAL COATING ON PLANT VIRUS NANOPARTICLES FOR BIOIMAGING AND THERAPEUTIC APPLICATIONS

(71) Applicant: THE REGENTS OF THE UNIVERSITY OF CALIFORNIA, Oakland, CA (US)

(72) Inventors: Jesse Vincent JOKERST, La Jolla, CA (US); Jiajing ZHOU, La Jolla, CA (US); Christian Isalomboto NKANGA, La Jolla, CA (US); Zhuohong WU, La Jolla, CA (US); Nicole F. STEINMETZ, La Jolla, CA (US)

(21) Appl. No.: 18/844,415

(22) PCT Filed: Mar. 9, 2023
(86) PCT No.: PCT/US2023/014892

§ 371 Date: Sep. 6, 2024

Related U.S. Application Data
(60) Provisional application No. 63/318,288, filed on Mar. 9, 2022.

(51) Int. Cl. A61K 47/69; A61K 33/243; A61K 41/00; A61K 49/00; A61K 49/22

(52) U.S. Cl. CPC A61K 47/6901; A61K 33/243; A61K 41/0052; A61K 49/0023; A61K 49/222; A61K 2121/00; A61K 2123/00; C12N 2770/00042; C12N 2770/18042; C12N 2770/26042

(57) ABSTRACT

A method for functionalizing plant viral nanoparticles (VNPs) includes selecting a plant VNP. Additionally, a metal ion and a phenolic compound that form a metal-phenolic network (MPN), and at least one functional component that adheres to the MPN are also selected. A nanohybrid structure is synthesized from a solution of the selected metal, the selected phenolic compound and the selected functional component such that the synthesized nanohybrid structure has an MPN coating encapsulating the plant VNP with the functional component being embedded in the MPN coating.

### CROSS-REFERENCE TO RELATED APPLICATION

This application is a National Phase in the United States of PCT/US2023/14892, filed Mar. 9, 2024, which claims the benefit of U.S. Provisional Application Ser. No. 63/318,288, filed Mar. 9, 2022, the contents of which are incorporated herein by reference.

### GOVERNMENT FUNDING

This invention was made with government support under CA202814, OD021821, and AG065776 awarded by the National Institutes of Health, and 1845683 awarded by the National Science Foundation. The government has certain rights in the invention.

### BACKGROUND

Plant viral nanoparticles (plant VNPs) have attracted immense interest in biomedical applications in the last decades including theranostics, drug, and gene delivery, and vaccine development owing to their multiple characteristics such as highly defined particle size and shape, amenability to genetic and chemical modifications and encapsulation strategies, as well as good scalability. Native plant VNPs exhibit variable immunogenicity and immunostimulatory properties for immunotherapeutic applications (e.g. in situ vaccination for cancer immunotherapy). Many efforts have been made to chemically or genetically modify plant for multiple desired applications. For instance, genetic modification can introduce unnatural amino acids as chemically addressable groups for orthogonal reaction or can create stabilizer that templates the formation of VNPs leading to functional protein overcoat or enzyme encapsulation. However, though elegant, these methods require elaborate coding of nucleotides into the viral genome. Physical techniques are promising strategies, such as encapsulation of small molecule antitumor drugs or inorganic nanoparticles into VNPs by disassembly and reassembly of the viral capsids modulated by electrostatic interactions. However, this approach in turn limits the scope of cargos where negatively-charged molecules, macromolecules, or nanoparticles are preferred. Another avenue are chemical-based strategies such as bioconjugation methods mediated by mild reactions (e.g. carbodiimide activation and click chemistry) have emerged as an important approach for virus functionalization. For example, therapeutic drugs, fluorescent dyes, and MRI contrast agents can be loaded onto VNPs via covalent attachment to the reactive amino acid residues on the exterior or interior surfaces of the viral capsids. Nevertheless, these modifications require multiple steps and tedious processing. Therefore, a simple and versatile strategy is of scientific and practical interest to functionalize plant VNPs.

Artificial bioaugmentation has recently attracted immense attention because it can impart functional properties to biomaterials. This strategy involves the exogenously or endogenously coupling of synthetic materials and biological components (e.g., biomacromolecules and living organisms) to afford resulting biohybrids with enhanced performances or new functions. Metal-organic materials, e.g., metal-organic frameworks (MOFs), are promising candidates for constructing exogenous functional matrixes or coatings on enzymes, living cells, and plant viruses, and therefore have been widely used for functional biohybrids. Metal-phenolic networks (MPNs) are formed via supramolecular interactions (i.e., chelation) between metal ions and natural polyphenols and have gained interest due to their easy synthesis, low toxicity, and high affinity to various bio-interfaces. For example, a simple yet dually functional MPN nanoshell was exogenously constructed on individual yeast cells. These cells were responsible for external stimuli and were degradable under certain conditions (e.g., pH).

### SUMMARY

In one aspect, a versatile supramolecular coating strategy for designing functional plant VNPs via metal-phenolic networks (MPNs) is presented. In one specific example, the disclosed method gives the plant viruses [e.g., tobacco mosaic virus (TMV), cowpea mosaic virus (CPMV), and potato virus X (PVX)] additional functionalities including photothermal transduction, photoacoustic imaging, and fluorescent labeling via different components in MPNs coating [i.e., complexes of tannic acid (TA), metal ions (e.g., Fe³⁺, Zr⁴⁺, or Gd³⁺), or fluorescent dyes (e.g., rhodamine 6G, thiazole orange)]. For example, using TMV as a viral substrate, by choosing Zr⁴⁺-TA, and rhodamine 6G, fluorescence is observed peaking at 555 nm; by choosing Fe³⁺-TA coating, the photothermal conversion efficiency was increased from 0.8% to 33.2%, and the photoacoustic performance was significantly improved with a limit of detection (LOD) of 17.7 μg·mL⁻¹. We further confirmed that TMV@Fe³⁺-TA nanohybrids show good cytocompatibility and excellent cell killing performance in photothermal therapy with 808-nm irradiation. These findings not only prove the practical benefits of this supramolecular coating for designing multifunctional and biocompatible plant viral nanoparticles but also bode well for using such materials in a variety of plant virus-based theranostic applications.

### BRIEF DESCRIPTION OF THE DRAWINGS

FIG. 1 is a schematic illustration of the synthesis of metal-phenolic networks (NPNs) coating on plant VNPs and biomedical applications thereof.

FIGS. 2a-2g illustrate the morphological, spectral, elemental characterizations of the Fe³⁺-TA coating on tobacco mosaic virus (TMV) nanoparticles.

FIGS. 3a-3c depict a miscellaneous combination of various metals, fluorescent dyes, and plant VNPs substrates for the formation of VNPs@MPN nanohybrids.

FIGS. 4a-4e illustrate the photothermal and photoacoustic performance of TMV@Fe³⁺-TA nanohybrids.

FIGS. 5a-5e illustrate the cell cytotoxicity and thermal ablation performance on SKOV3 using TMV@Fe³⁺-TA nanohybrids.

FIGS. 6a-6d show representative molecular structures.

FIGS. 7(a)(1)-7(a)(3) and 7(b)(1)-7(b)(3) show characterization data of the prepared VNPs.

FIGS. 8a-8e show the optimization of the morphology of TMV@Fe³⁺-TA. TEM images showing the Fe³⁺-TA coating morphology on TMV in final solutions with different concentrations.

FIGS. 9a-9b depict results of serial dilution proving dispersity of TMV@Fe³⁺-TA.

FIGS. 10a-10b illustrate the Fe³⁺-TA coating on TMV by EDS linear scan.

FIGS. 11a-11b show UV-Vis spectra of Gd³⁺-TA and Zr⁴⁺-TA coatings on TMV.

FIG. 12 shows the size distribution of metal-dependent TMV@MPN nanohybrids.

FIG. 13 shows photographs of various MPN coatings on TMV nanoparticles.

FIGS. 14a-14b shows data estimating the fluorescence performance of TMV@Zr⁴⁺-TA-Rh6G nanohybrids. FIGS. 15a-15c show the size distribution of various VNPs before and after Fe³⁺-TA coating.

FIGS. 16a1-16a2, 16b1-16b2 and 16c1-16c3 show magnified TEM images to show Fe³⁺-TA coating on VNPs.

FIGS. 17a-17c show an SDS-PAGE analysis of the viral coat proteins (CPs) from VNPs@Fe³⁺-TA vs. wild type VNPs.

FIG. 18 shows power-dependent temperature profiles of TMV@Fe³⁺-TA.

FIGS. 19a-19b show data for calculating the photothermal conversion efficiency. 24 FIGS. 20a and 20b1-20b2 show a photothermal stability assessment by TEM.

FIG. 21 shows the photoacoustic spectrum of TMV@Fe³⁺-TA.

FIG. 22 shows a linear analysis of TMV@Fe³⁺-TA at different concentrations.

FIG. 23 shows the limit of detection (LOD) of PA.

FIG. 24 shows calcein AM and PI-stained SKOV3 cells.

FIGS. 25a-25d show a disk diffusion assay for an anti-bacterial test.

### DETAILED DESCRIPTION

### Introduction

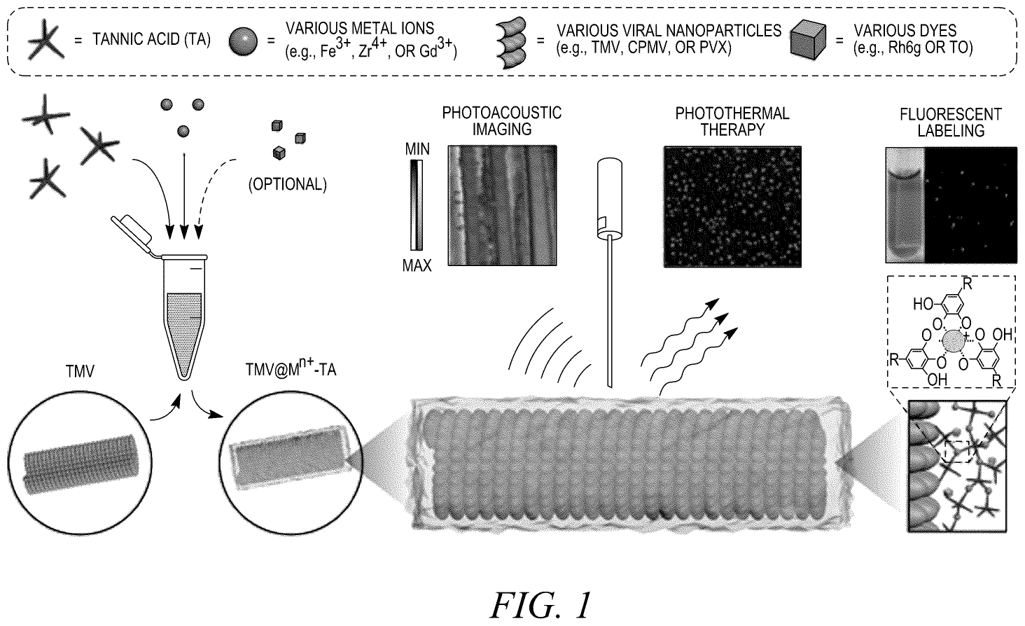

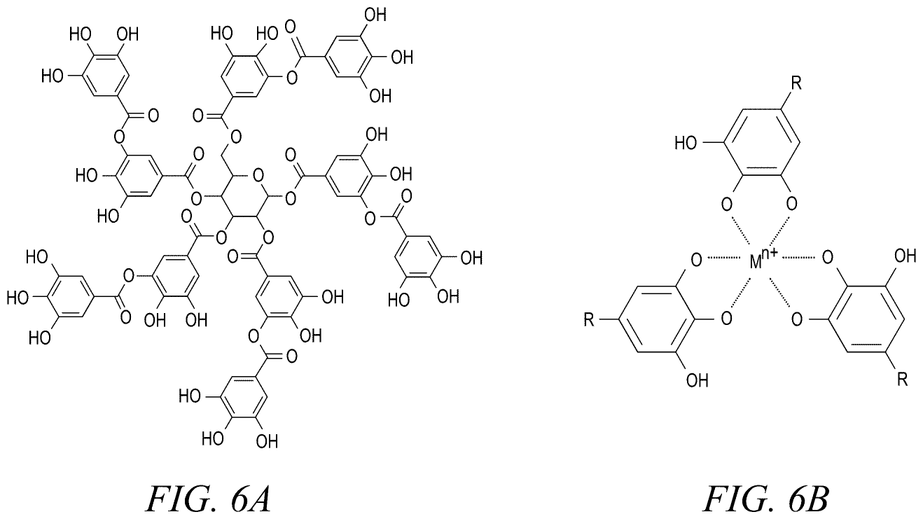

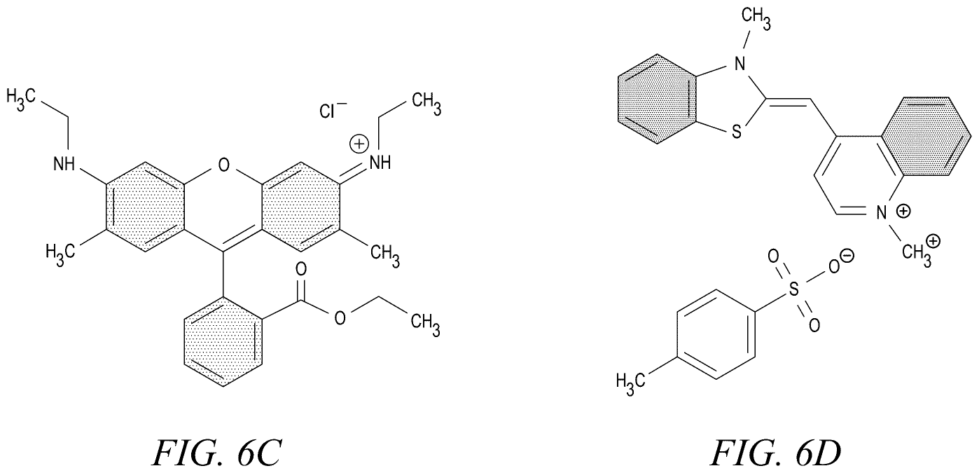

In this work, we describe a simple and versatile strategy to modify VNPs using MPNs for multiplex theranostic purposes including photoacoustic imaging (PAI), photothermal therapy (PTT), and fluorescent labeling (Scheme 1). Specifically, metal ions (e.g., Fe³⁺, Gd³⁺, and Zr⁴⁺) and tannic acid (TA) form MPN coatings with add-on fluorescence (i.e., by embedding fluorescent dyes into the MPN coatings) by exploiting the adherent nature of phenolic moieties (FIG. 6) to various metal ions, aromatic dyes, and proteins. FIG. 1 is a schematic of the synthesis of metal-phenolic networks (MPNs) coating on plant VNPs and biomedical applications. The phenolic-based coating enables the integration of photoacoustic signals, photothermal transduction, and fluorescent labeling into VNPs (e.g., TMV) through an optional selection of components. In the dashed box, R represents the remainder of the TA molecules, and Mⁿ⁺ represents the central metal ions with the oxidation number of n.

To demonstrate the concept, the strategy was validated by using a tobacco mosaic virus (TMV)—a rod-shaped nucleoprotein assembly measuring 300×18 nm with a 4 nm wide accessible internal channel. The proposed multifunctional design fulfills the following purposes: first, by incorporating fluorescent dyes, fluorescent labeling facilitates the direct tracking of TMV via fluorescence microscopy, such as intravital imaging, and can serve as a tag for ex vivo quantification, e.g. for biodistribution and clearance studies. Then, the TMV can afford the PA signal when coated with complexes that induce ligand-to-metal charge-transfer (LMCT) bands (i.e., Fe³⁺-TA). PA imaging is a non-invasive imaging approach that relies on acoustic waves generated by biological tissues or contrast agents upon absorbing light energy; PA imaging allows for a deeper imaging penetration up to ˜5.2 cm in the NIR-I window (650-950 nm). Finally, the system offers PTT with minimal invasiveness and precise spatial-temporal control. MPN-based materials have been reported to show potentials for PA imaging and PTT.

Compared to previous strategies on virus modification, our technique has several advantages. First, the synthesis of MPN coatings on VNPs is cost-effective and environmental-friendly, which means it can be finished in a few seconds at room temperature without the need of organic solvents. Second, miscellaneous selection of functional components (i.e., metal ions and fluorescent dyes) provides flexibility for clinical scenarios, such as using Gd³⁺ as metal ion sources for MRI signal enhancement or near-infrared dyes for imaging-guided therapy. Third, we also demonstrate the robustness of this approach and show that this strategy can be applied to other plant viruses of different shapes, e.g., the icosahedral cowpea mosaic virus (CPMV) and the filamentous potato virus X (PVX), which suggests the generalized nature of this method. Relevant studies are compared in supplementary information (Table 1). We envision that this system can advance VNP-based applications and also facilitate the development of artificial bioaugmentation for a wider range of biomaterials including the many protein-based nanoparticles either naturally derived or programmed through origami.

TABLE 1
Relevant choice of substrate, functional materials,
and the corresponding applications

| | Functional | | |
|---|---|---|---|
| Substrate(s) | materials | Application(s) | Ref. |
| Tobacco mosaic | Fe³⁺/Gd³⁺/ | Fluorescent labelling; | This |
| virus; cowpea | Zr⁴⁺-TA-Rh6G/TO | photoacoustic imaging; | work |
| mosaic virus; | | photothermal therapy | |
| potato virus X | | | |
| Tobacco mosaic | ZIF-8 | Protective coating; | 6 |
| virus | | slow release. | |
| Tobacco mosaic | ZIF-8 | template-directed | 7 |
| virus | | synthesis | |
| Tobacco mosaic | Gd(DOTA)- | Magnetic resonance | 3 |
| virus | azide; | imaging; photoacoustic | |
| | polydopamine | imaging; photothermal | |
| | | therapy | |
| Brome mosaic | Fe³⁺-TA | Protective barrier to | 8 |
| virus | | dehydration | |
| Enzymes | PCN-333(Al) | enzyme encapsulation | 9 |
| Proteins, | ZIF-8 | Protective coating; | 10 |
| enzymes, | | slow release | |
| and DNA | | | |
| Proteinosomes | Fe³⁺-TA | Protective coating | 11 |
| Yeast cell | ZIF-8 | Protective coating | 12 |
| Yeast cell | Fe³⁺-TA | UV and ROS protection | 13 |
| Mammalian | Fe³⁺-TA | Protective coating; | 14 |
| cells | | cell growth control | |

Abbreviation:
ZIF-8, zeolitic imidazolate framework-8;
Gd, gadolinium;
DOTA, monoamide-1,4,7,10-tetraazacyclododecane-N-N′-N″-N″′-tetraacetic acid;
PCN, porous coordination network

### EXAMPLES AND ANALYSIS

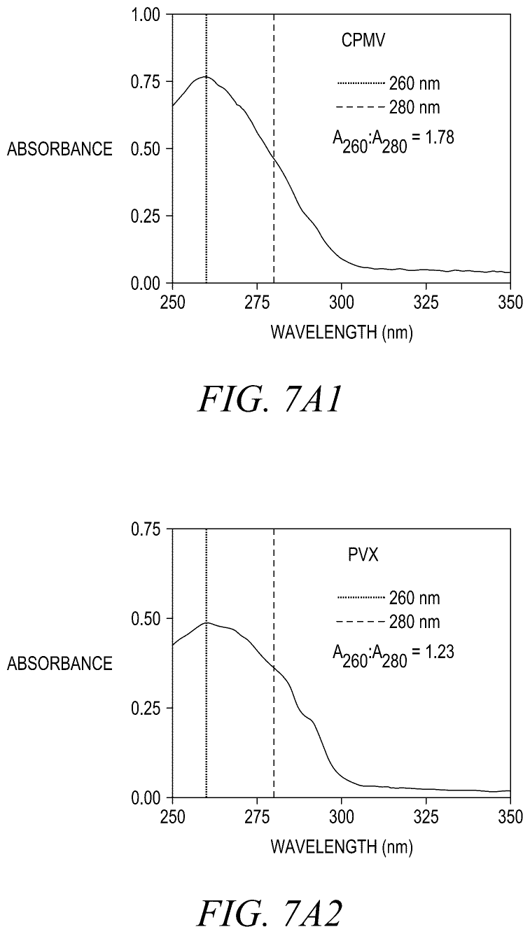

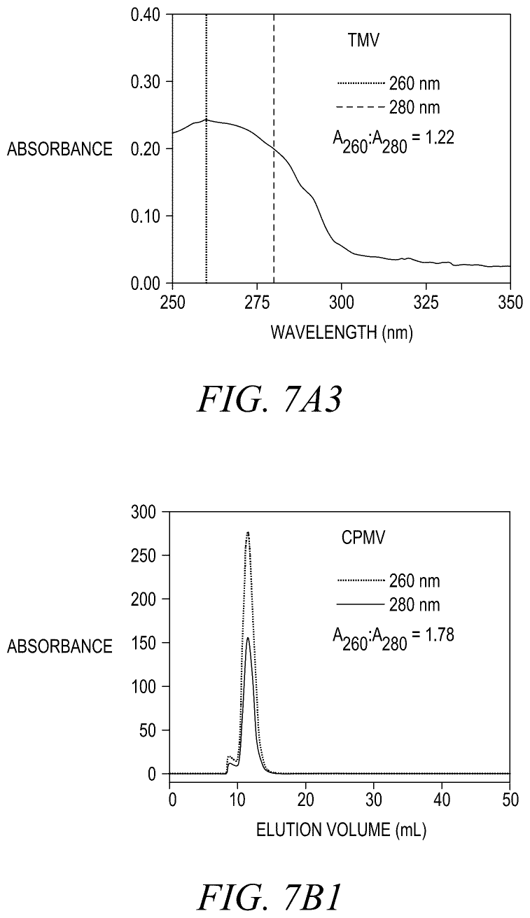

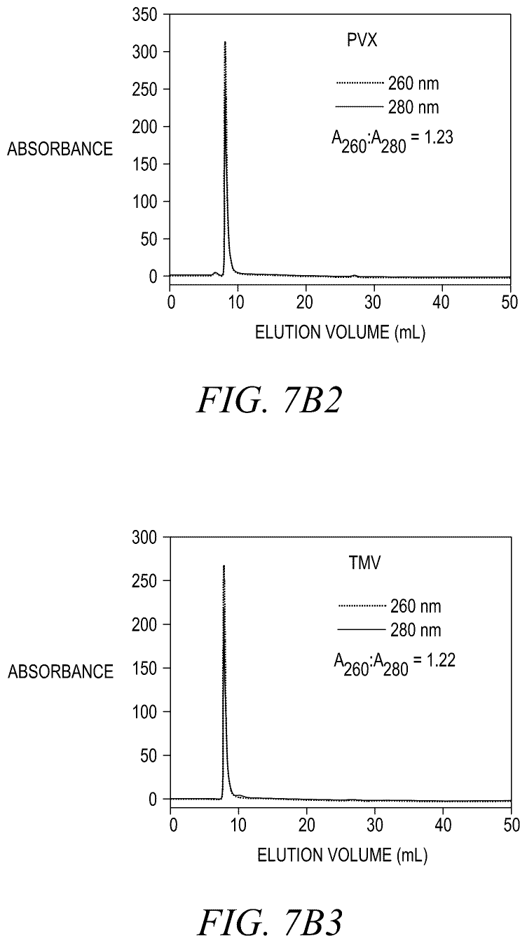

Plant viruses were propagated in and purified from the leaves of different plant species using established protocols: CPMV particles were extracted from Vigna unguiculata; PVX and TMV were isolated from Nicotiana benthamiana. The concentrations of purified particles were determined by UV spectrometry at the maximum absorption wavelength of 260 nm for all VNPs (FIG. 7a), using the Beer-Lambert law with molar extinction coefficients of 8.1 mL mg⁻¹ cm⁻¹ (ε_{CPMV}); 2.97 mL mg⁻¹ cm⁻¹ (ε_{PVX}); and 3.0 mL·mg⁻¹·cm⁻¹ (ε_{TMV}). Since the UV absorbance of viral RNA peaks at 260 nm, and viral coat protein peaks at 280 nm, the ratios between the absorbances at 260 nm and 280 nm (A₂₆₀:A₂₈₀) were found to be 1.78 for CPMV (expected 1.7-1.9); 1.23 for PVX (expected 1.1-1.3) and 1.22 for TMV (expected value 1.0-1.2), indicating acceptable purity of the produced VNPs. The analysis by size-exclusion fast protein liquid chromatography (FPLC) exhibited a single peak elution profile (FIG. 7b). The co-elution of the viral constructs showed the integrity of the prepared particles, with negligible broken or aggregates. To optimize the coating protocol, we selected TMV particles as a model VNP; TMV is the most robust platform and previous data demonstrated the potential of TMV nano-platforms for the development of multifunctional theranostic reagents.

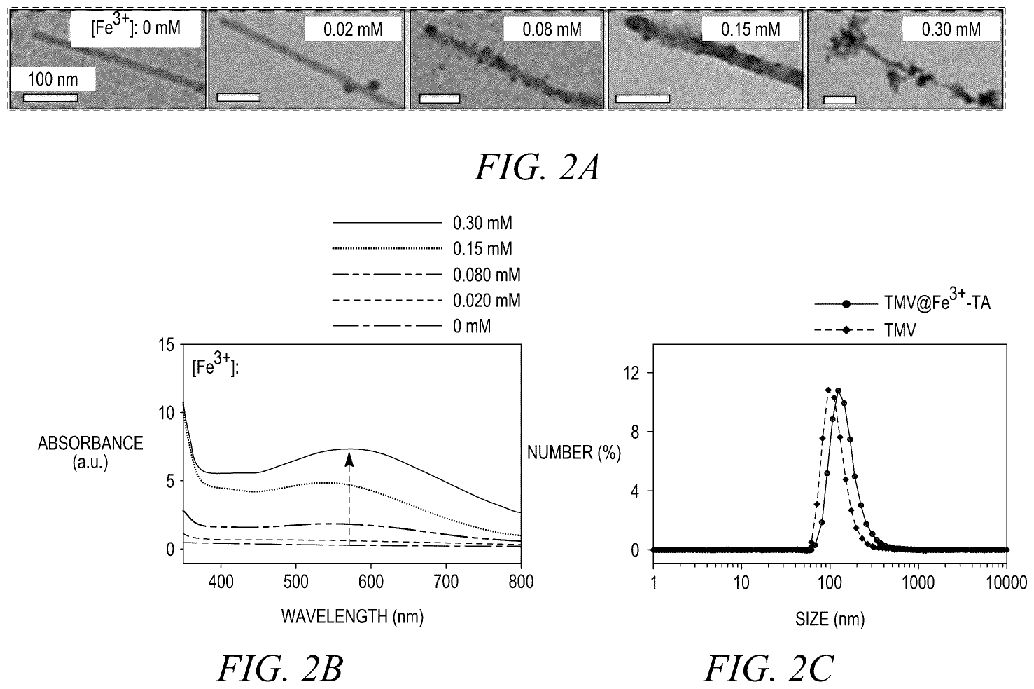

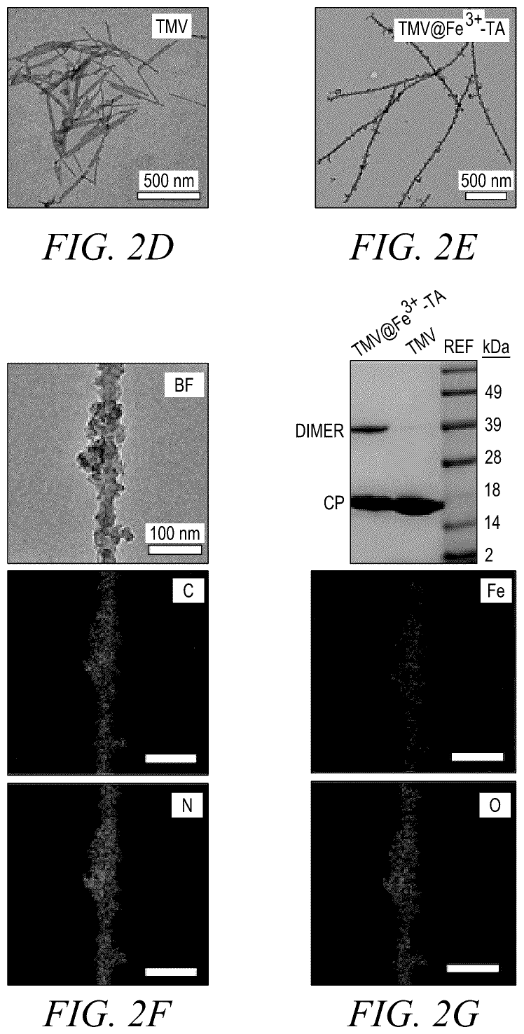

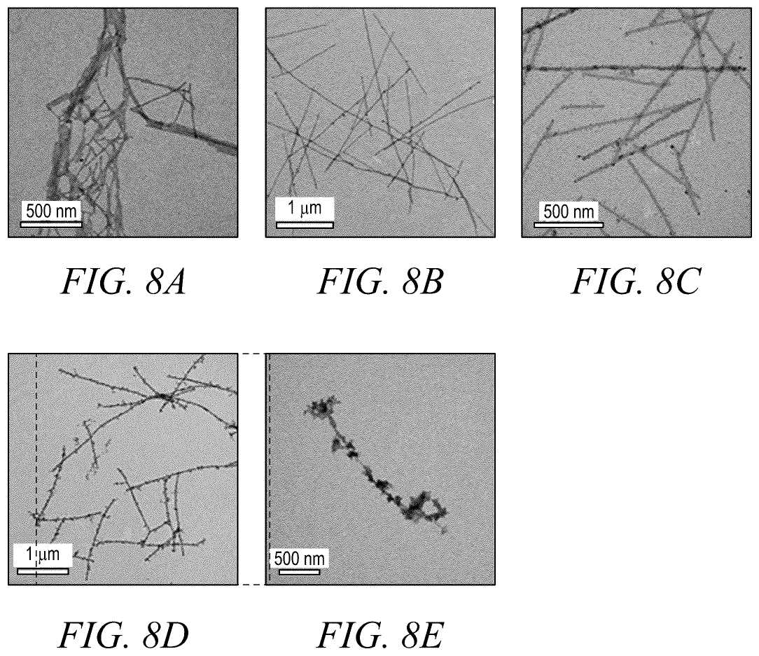

The native TMV nanoparticles were coated with MPN (e.g., Fe³⁺-TA) via a one-step method. Specifically, reagents were sequentially added to yield the final concentrations of TMV suspension (0.22 mg·ml⁻¹), TA (0.052 mM), Fe³⁺ solution (0.15 mM), and TRIS buffer (10 mM, pH 8.5). The reaction can be finished in seconds. The TMV suspension remained colorless after mixing with TA solution and immediately turned to dark blue upon the addition of Fe³⁺ and TRIS buffer due to the formation of Fe³⁺-TA complexes on the TMV nanoparticles. The molar ratio of Fe³⁺ and TA was set to 3:1, which is an optimized stoichiometry for the Fe³⁺-TA complexes formation via chelation. The final concentrations of Fe³⁺ varied from 0-0.3 mM and the optimized formulation was adopted based on the morphology evaluated by transmission electron microscopy (TEM), which is shown in FIG. 2a (see also FIG. 8) and the coating thickness characterized by UV-Vis spectra. It should be noted that scale bars are 100 nm in FIG. 2(a) as well as in FIG. 2(f), discussed below. FIG. 2b shows the UV-Vis spectra of TMV@Fe³⁺-TA with different concentrations of Fe³⁺ varying from 0 to 0.30 mM (indicated by the arrow), respectively. Formulations in FIGS. 2a and 2b were labeled by the concentration of Fe³⁺ only due to the fixed stoichiometry of Fe³⁺ to TA, which is 3:1. The formulation of Fe³⁺ (0.15 mM) and TA (0.050 mM) was adopted as the optimized formulation.

To characterize the morphology, initially, TMV particles with no coating (0 mM Fe³⁺) exhibited a clean and smooth surface. Similarly, the TMV@Fe³⁺-TA (0.02 mM Fe³⁺) displayed no obvious coating on the particle's surface but there were knot-like structures. At higher concentrations of Fe³⁺ and TA, TMV@Fe³⁺-TA (0.08 mM Fe³⁺) became rougher with dark clumps. In contrast, TMV@Fe³⁺-TA (0.15 mM Fe³¹) exhibited a smooth and dense coating. When excess precursors were added (0.30 mM Fe³⁺), overloaded Fe³⁺-TA complexes were loosely attached to TMV's surface. Increased concentrations of precursors led to TMV@Fe³⁺-TA nanohybrids with a larger absorbance from 350 to 800 nm; the peak was at 570 nm (see FIG. 2b). The stronger ligand-to-metal charge-transfer (LMCT) bands indicate thicker MPN coatings via the cross-linking of TA by Fe³⁺ ions. Based on the results from TEM and UV-Vis spectra, the formulation of [Fe³⁺]:[TA]=0.15:0.050 in mM was adopted as the optimized recipe.

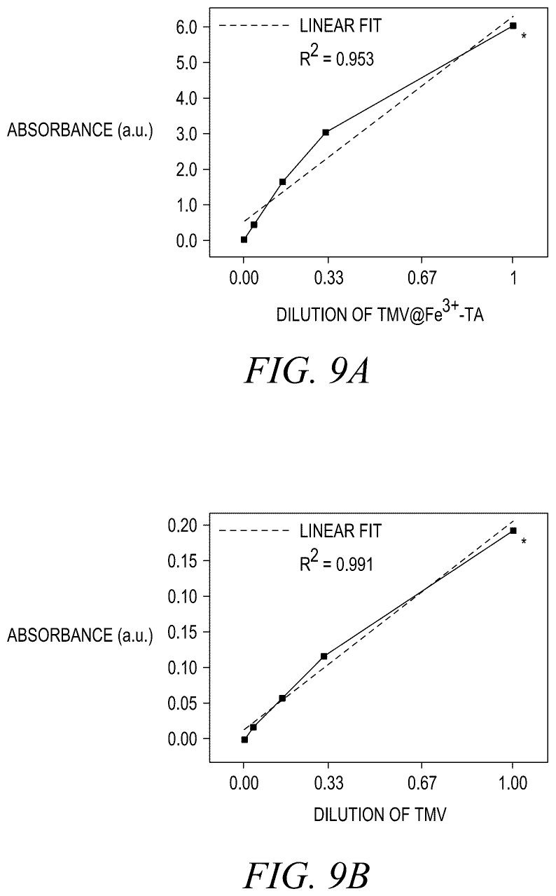

Further, to evaluate the optimized TMV@Fe³⁺-TA nanohybrids, dynamic light scattering (DLS) data showed that the formation of Fe³⁺-TA coating on TMV significantly shifted the surface zeta potential values from −19.2±5.8 to −30.3±11.3 mV (in PBS buffer) and slightly shifted the particles size distribution peak from 99 nm to 119 nm, before and after coating, indicating the formation of coating and absence of noticeable aggregation. FIG. 2c shows the DLS data, which depicts the size distributions of TMV and TMV@Fe³⁺-TA. Note that though the absolute values of size results from DLS are meant for spherical particles, here is used for relative comparison to prove the size change caused by coating and the good dispersity of nanohybrids in water-based buffer. Also, a serial dilution on TMV@Fe³⁺-TA nanohybrids (R²=0.953) also demonstrated its good dispersibility and homogeneity in DI water (FIG. 9). Further, the TEM images compared the morphology of TMV before and after Fe³⁺-TA coating. FIGS. 2d and 2e show representative TEM images of bare TMV nanoparticles and TMV@Fe³⁺-TA nanohybrids, respectively. Notably, the elongation of coated nanohybrids is attributed to the addition of tannic acid. In an acidic environment, the head-to-tail assembly of TMV is highly favored by minimizing the electrostatic repulsion between the carboxylic residues at the lateral area of adjunct TMV particles as well as hydrophobic interactions.

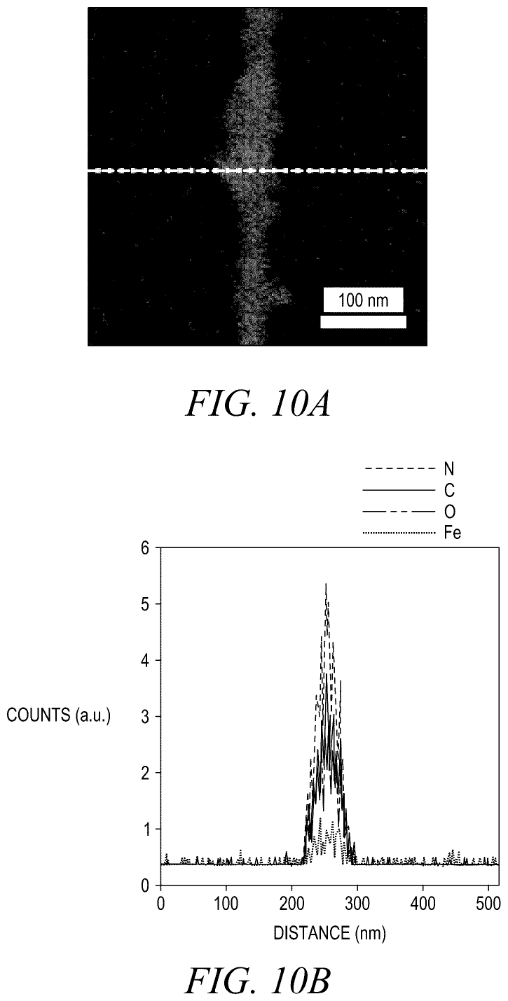

Energy dispersive X-ray (EDX) elemental mapping and linear scanning (FIG. 10) further validated the presence of C, N, and O signals from TMV and TA as well as Fe signal from the formed Fe³⁺-TA complexes. FIG. 2f shows the EDX elemental mapping of the TMV@Fe³⁺-TA nanohybrids. The signal of Fe verified the formation of Fe³⁺-TA complexes on TMV. Sodium dodecyl sulfate-polyacrylamide gel electrophoresis (SDS-PAGE) was used to analyze the TMV coat protein (CP). FIG. 2g shows a SDS-PAGE analysis of the viral capsid protein (CP) from wild-type TMV particles and TMV@Fe³⁺-TA nanohybrids. CPs from both coated and uncoated TMV displayed identical electrophoretic profiles, which indicates that their chemical composition remained unchanged after coating. The CP from TMV@Fe³⁺-TA nanohybrids exhibited a similar electrophoretic profile compared to native uncoated TMV with the expected molecular weights of ˜18 kDa, suggesting that the CP is not covalently modified upon coating with Fe³⁺-TA complexes—as expected. Notably, TMV@Fe³⁺-TA exhibited an additional band at ˜39 kDa corresponding to the CP dimers, which was also previously observed when coating TMV with polydopamine, perhaps reflecting the intertwining effect of the MPN matrix. Overall, the assembly of Fe³⁺-TA complexes on TMV is therefore possibly driven solely by non-covalent intermolecular interactions, such as hydrophobic interactions, hydrogen bonding, and ionic interactions.

In conclusion, we optimized the coating protocol for VNPs@MPN by selecting TMV and Fe³⁺-TA as models. The nanohybrids with a consistent morphology could be prepared using TMV of 0.20 mg·mL⁻¹ and [Fe³⁺]:[TA]=0.15:0.050 in mM, and we used this formulation for a broader range of nanohybrids and further applications.

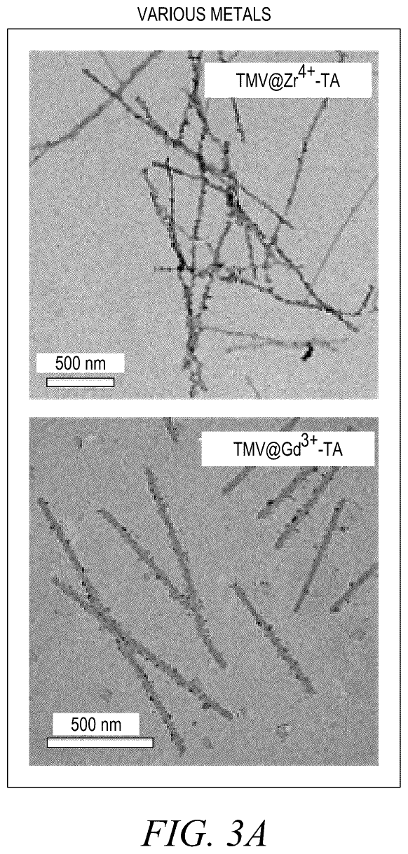

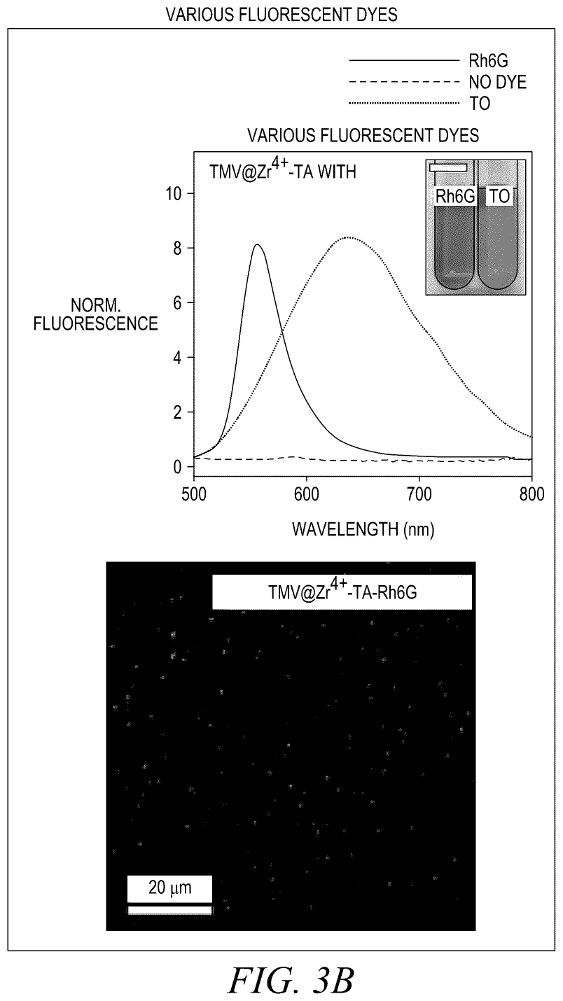

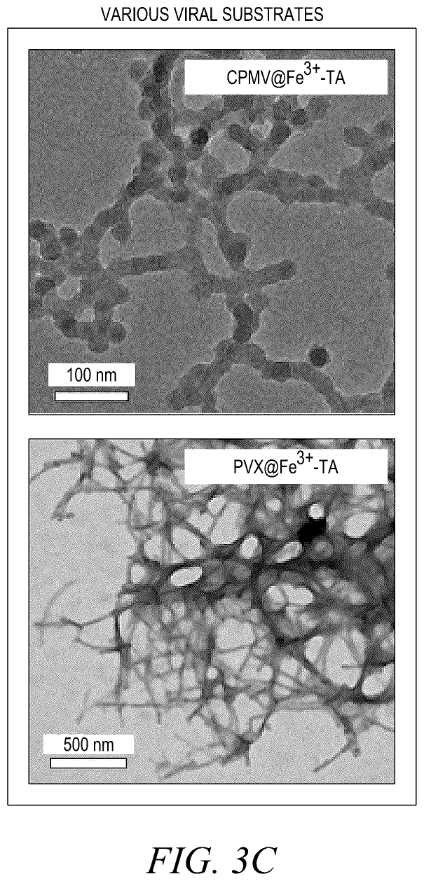

Next, to expand the scope of the coating method, by using the same formulation optimized above, modular assembly of VNPs@MPN can be achieved with the miscellaneous selection of functional building blocks, including metal ions (e.g., Gd³⁺ and Zr⁴⁺), fluorescent dyes [e.g., Rhodamine 6G (Rh6G) and Thiazole Orange (TO)](FIG. 6), and different plant VNPs substrates (e.g., PVX and CPMV). First, various metals coordinate with TA via chelation, and physiochemistry of the resulting complexes is largely dependent on the type of metal ion and the corresponding oxidation number. For example, Fe³⁺-based MPN was well-demonstrated to be highly biocompatible; tetra-valent metal ions (e.g., Zr⁴⁺) were generally more stable and thicker than di- or tri-coordinating ions; and Gd³⁺ ions can afford the MPN with MRI signals in clinical diagnosis. Representative TEM images showed TMV nanohybrids enabled by MPN with Zr⁴⁺ and Gd³⁺, respectively. FIG. 3a shows TEM images of TMV@Zr⁴⁺-TA and TMV@Gd³⁺-TA. [Metals]=0.15 mM; [TA]=0.050 mM.

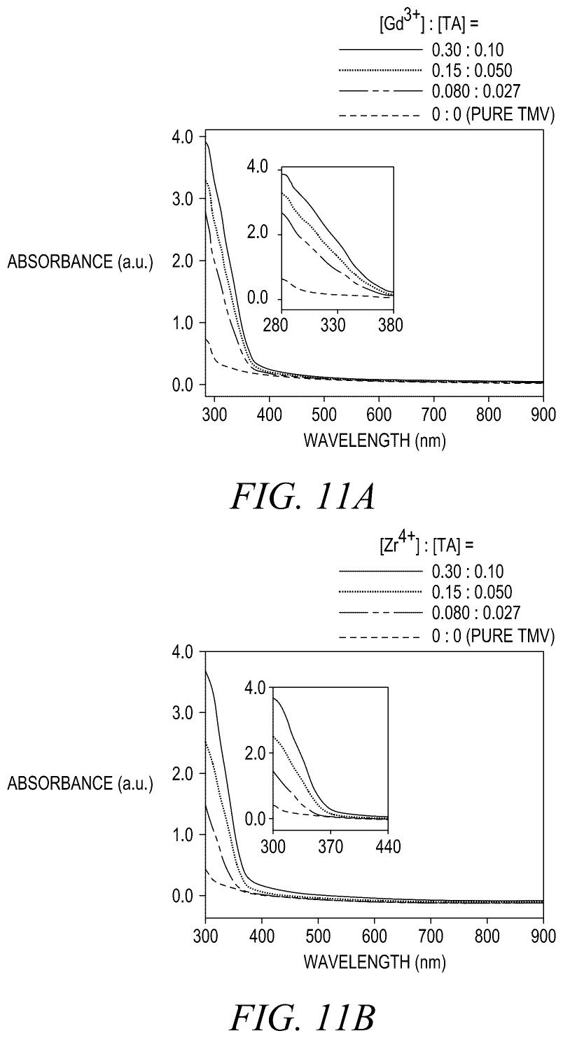

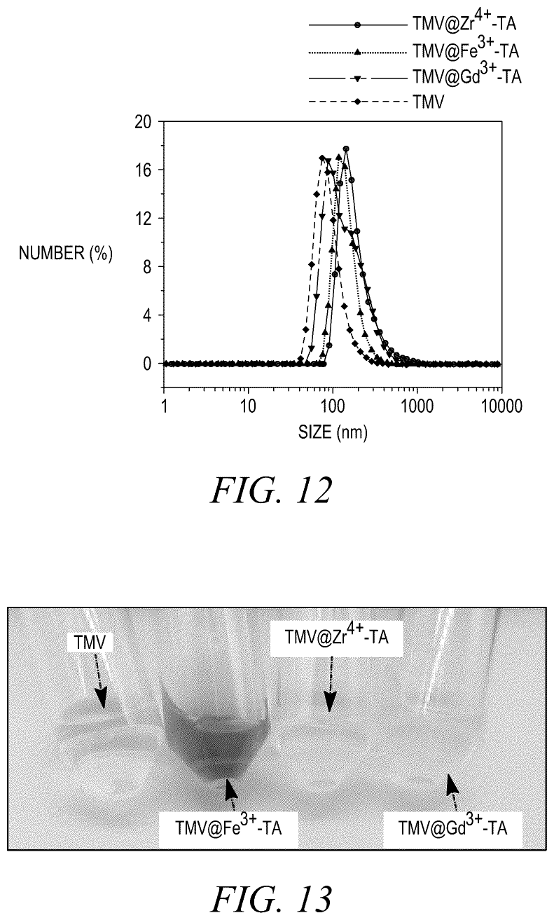

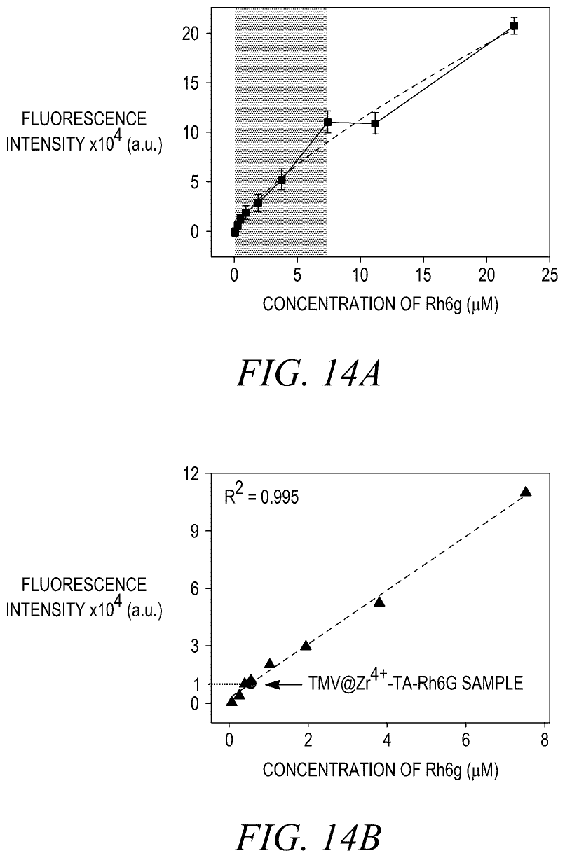

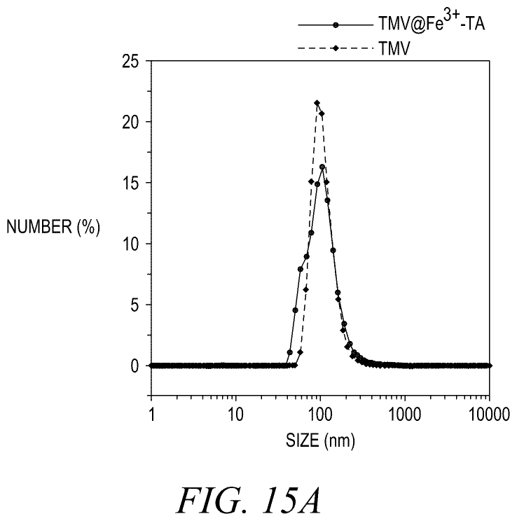

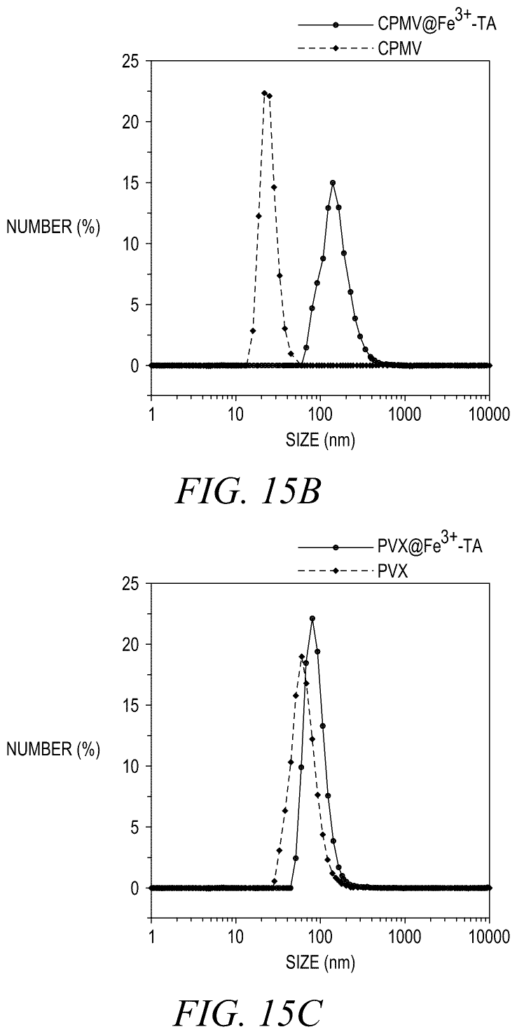

UV-Vis spectra demonstrated increased absorbance in the range of 300-380 nm for particles coated with increasing concentrations of Zr⁴⁺ and Gd³⁺ (FIG. 11). DLS results (FIG. 12) showed that the sizes of TMV@Zr⁴⁺-TA particles were larger than TMV@Fe³⁺-TA and TMV@Gd³⁺-TA particles, which can be explained by the higher coordination numbers of Zr⁴⁺. Further, various aromatic fluorescent dyes interact with TA by π-π stacking and cation-π interactions. This inspired us to investigate the incorporation of fluorescent dyes (i.e., Rh6G and TO) into the biohybrids. Zr⁴⁺-based MPN was chosen for fluorescent labeling due to its relatively lower extinction compared to the Fe³⁺-TA coating (FIG. 13). In FIG. 3b, the upper panel shows normalized fluorescence spectra of suspensions of TMV@Zr⁴⁺-TA loaded with Rh6G and TO. The inset in FIG. 3b is a photograph of the corresponding suspensions. The lower panel shows TMV@Zr⁴⁺-TA with Rh6G in DI water excited at 488 nm. [Dyes]=3 M; [Zr⁴+]=0.15 mM; [TA]=0.050 mM. The scale is 3 mm in the inset of the upper panel in FIG. 3b. The fluorescence spectra showed that the TMV@Zr⁴⁺-TA nanohybrids were loaded with Rh6G or TO; fluorescence microscopy confirmed green fluorescence from TMV@Zr⁴⁺-TA-Rh6G excited at 488 nm. To estimate the fluorescence performance of such TMV@Zr⁴⁺-TA-Rh6G nanohybrids, linear regression (R²=0.995) was plotted; the fluorescence intensity is roughly equivalent to 0.49 μM free Rh6G solution in DI water when 3.0 μM Rh6G was used to synthesize such nanohybrids (FIG. 14). The loss of fluorescent intensity was also observed when loading rhodamine B on polystyrene, reflecting the quenching effect of TA. The fluorescent MPNs complexes with various dyes have proven to be stable on polystyrene at pH from 1-8 and in complex biological media incubating for 24 hours, which are attributed to the dominant π interactions between the dyes and phenolic motifs.

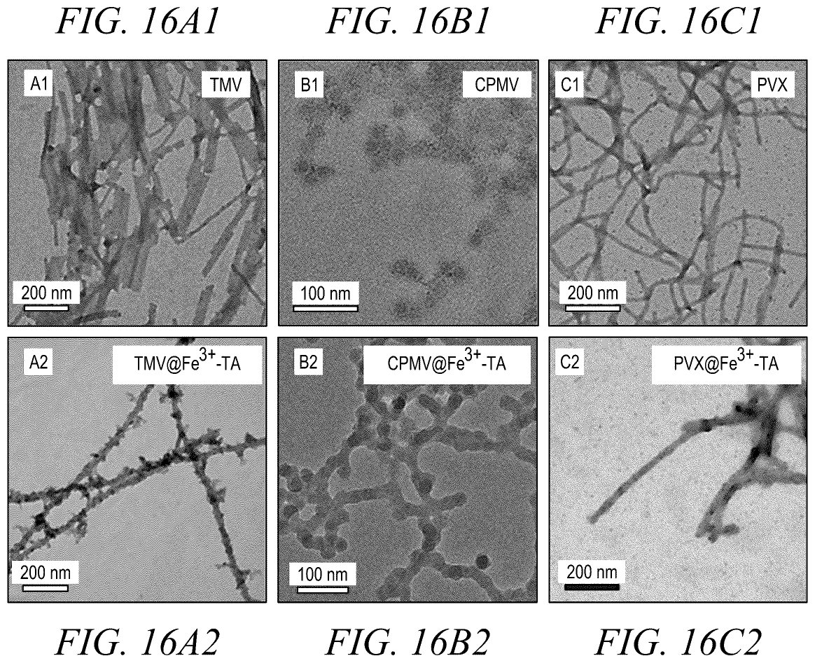

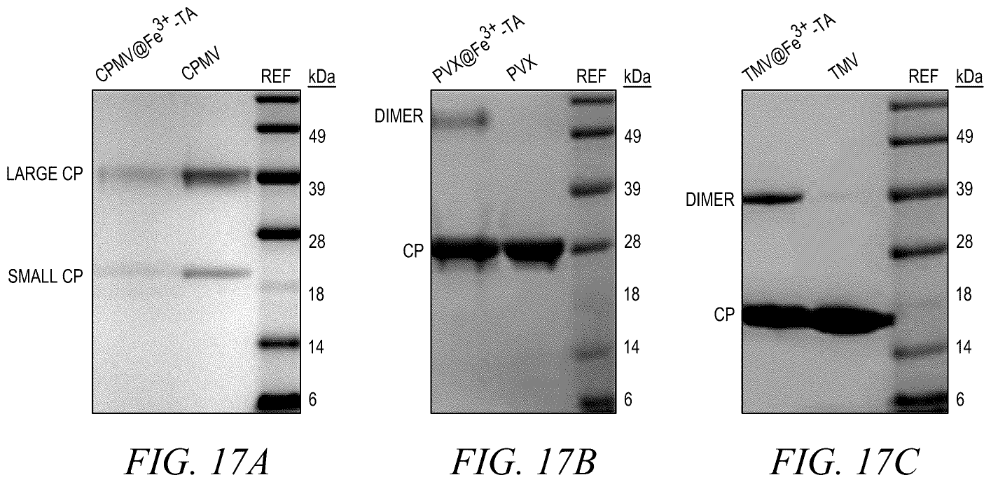

Finally, we investigated the coating on VNPs of different shapes, i.e., PVX and CPMV. PVX is an elongated filamentous and flexible VNPs that has a high aspect ratio (515×13 nm), while CPMV is an icosahedral virus with a diameter of 30 nm. Using the same coating formulation that produces TMV@Fe³⁺-TA (i.e., 0.15 mM Fe³⁺, 0.15 mM TA, and 0.20 mM VNPs), successful designs of PVX@Fe³⁺-TA and CPMV@Fe³⁺-TA were confirmed by TEM. FIG. 3c shows TEM images of icosahedral CPMV@Fe³⁺-TA (top panel) and filamentous PVX@Fe³⁺-TA (bottom panel). [VNPs]=0.20 mg·mL⁻¹; [Fe³⁺]=0.15 mM; [TA]=0.050 mM. In addition, DLS data showed that the formation of Fe³⁺-TA coating on CPMV and PVX resulted in increased particle sizes when compared to the native VNPs (FIG. 15, Table 3). The values of surface zeta potentials were more negative (Table 2). Side-by-side TEM images of all coated and uncoated viruses showed distinct coatings that are in high contrast (FIG. 16). These observations indicate that Fe³⁺-TA coatings successfully formed on different VNPs. SDS-PAGE was used to analyze the CPs from uncoated VNPs (i.e., CPMV and PVX) and VNPs@Fe³⁺-TA. Irrespective of the type of VNPs, the CPs of the coated VNPs described similar electrophoretic profiles compared to the uncoated CP (FIG. 17). This is consistent with the results obtained for TMV and indicated no breakage of protein and unchanged chemical compositions due to the mild reaction condition and non-covalent nature of MPN, as described in FIG. 2g. Collectively, we demonstrated miscellaneous combinations available for the formation of VNPs@MPN nanohybrids.

TABLE 2
Comparison of VNP size, shapes and zeta potential
before and after coating with Fe³⁺-TA

| | | | Zeta potential | Zeta potential |
|---|---|---|---|---|
| Plant | Size | | (Before coating) | (After coating) |
| virus | (nm) | Capsid shape | (mV) | (mV) |
| TMV | 18 × 300 | Helical, rigid | −19.2 ± 5.8 | −30.3 ± 11.3 |
| PVX | 13 × 515 | Helical, | −21.0 ± 6.9 | −35.7 ± 8.6 |
| | | filamentous | | |
| CPMV | 30 | icosahedral | −11.0 ± 6.3 | −34.0 ± 6.3 |

Date of VNP sizes, shapes are from reference.

TABLE 3
Comparison of VNP size distribution and
PDI before and after coating with Fe³⁺-TA

| | DLS | PDI | DLS | PDI |
|---|---|---|---|---|
| Plant | (Before | (Before | (After | (After |
| virus | coating) | coating) | coating) | coating) |
| TMV | 109.1 ± 48.3 | 0.188 | 110.7 ± 41.2 | 0.198 |
| [Fe³⁺]:[TA] | | | [0.15]:[0.05] | |
| PVX | 65.8 ± 25.0 | 0.277 | 89.6 ± 31.1 | 0.439 |
| [Fe³⁺]:[TA] | | | [0.15]:[0.05] | |
| CPMV | 25.8 ± 5.6 | 0.248 | 33.8 ± 10.7 | 0.280 |
| [Fe³⁺]:[TA] | | | [0.075]:[0.025] | |

*0.22 mg · mL⁻¹ VNP is used for all synthesis.
All data acquired by DLS, n = 3.
Unit: nm for DLS, mM for final reagent concentrations.
PDI: polydispersity index.

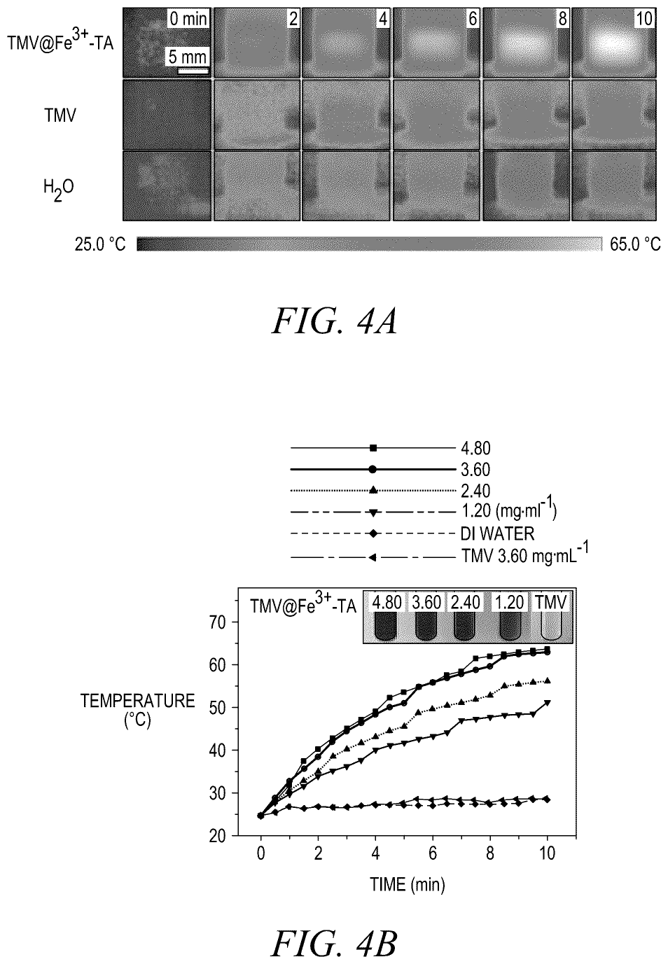

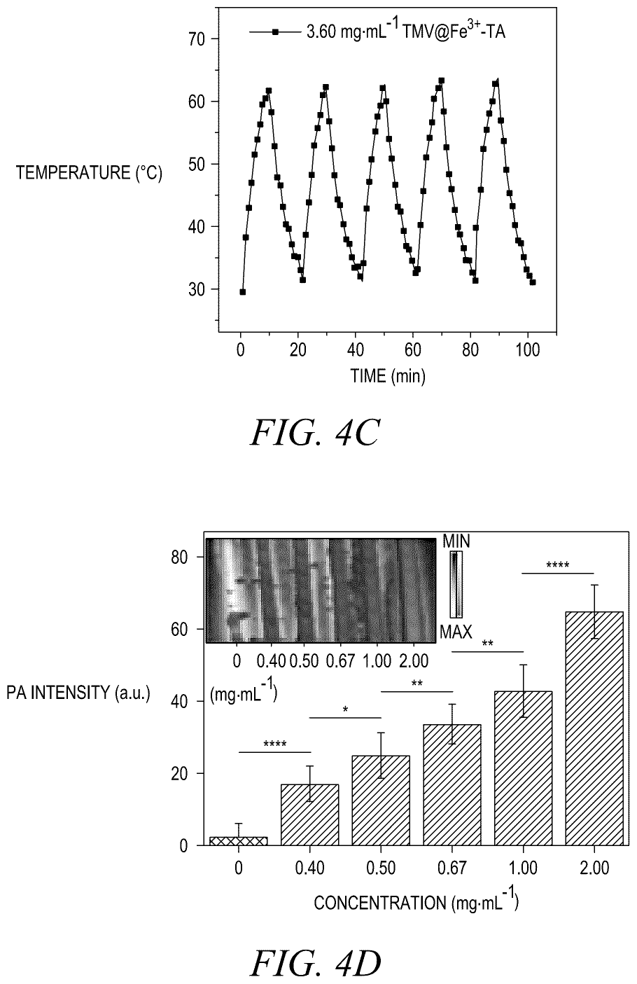

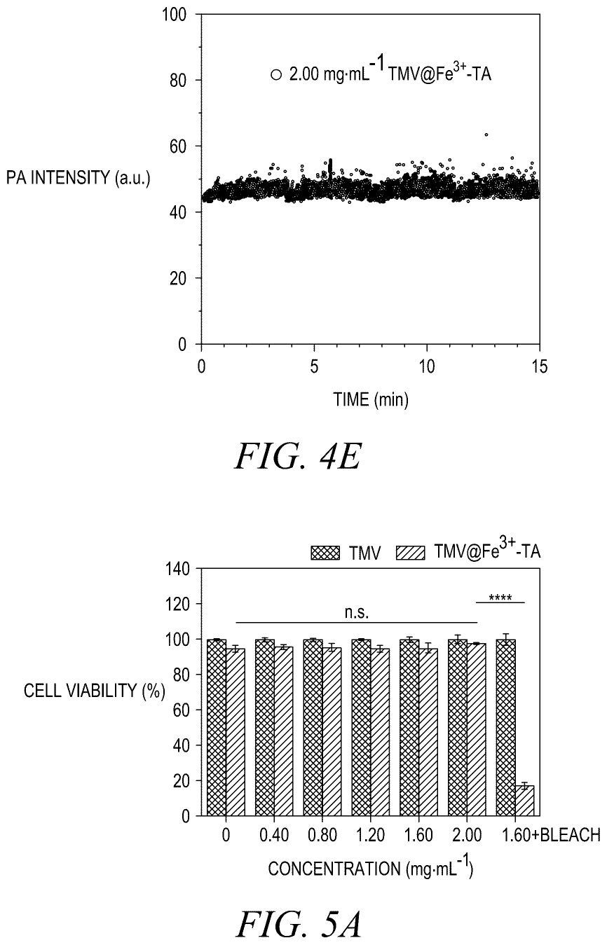

Among a different selection of components for practical applications, the strong extinction of LMCT of Fe³⁺-TA implies photo-mediated applications such as PTT performance and PA imaging. The color of Fe³⁺-TA complexes is bluish-black because the complexes strongly absorb light in the visible and NIR-I region. This motivated us to investigate the photothermal conversion capability imparted by the Fe³⁺-TA coating. The temperature profiles of TMV@Fe³⁺-TA (3.60 mg·mL⁻¹), TMV (3.60 mg·mL⁻¹), and DI water irradiated with 808 nm laser were monitored using a near-infrared (NIR) camera. FIG. 4(a) shows thermal images of TMV@Fe³⁺-TA (3.60 mg·mL⁻¹), pure TMV (3.60 mg·mL⁻¹), and DI water upon 808 nm irradiation for 10 min. FIG. 4(b) shows temperature profiles of TMV@Fe³⁺-TA suspensions irradiated by 808 nm laser (1.0 W·cm⁻²). The inset is a photograph of TMV@Fe³⁺-TA at different concentrations (from left to right: 4.80, 3.60, 2.40, and 1.20 mg·mL-1 TMV@Fe³⁺-TA, and 2.00 mg·mL⁻¹ pure TMV.

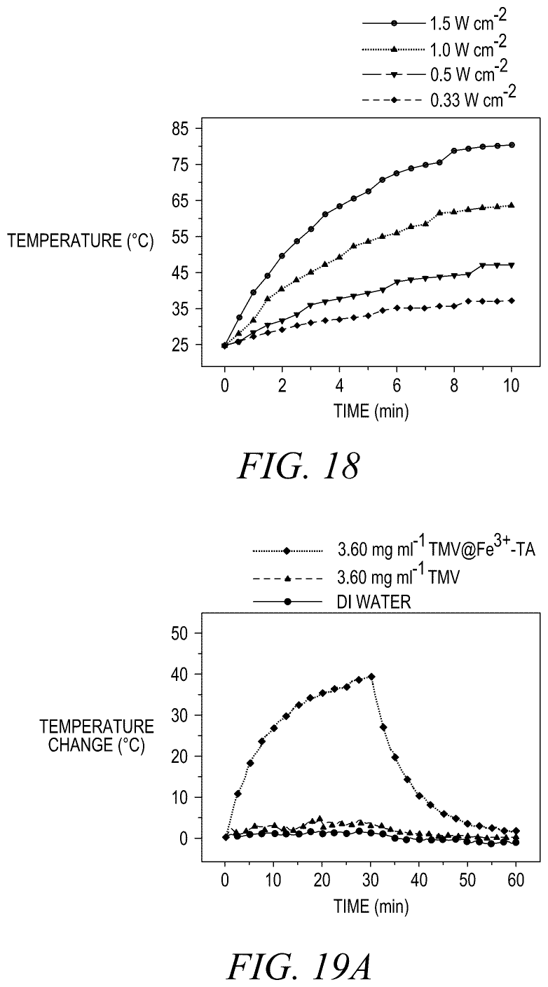

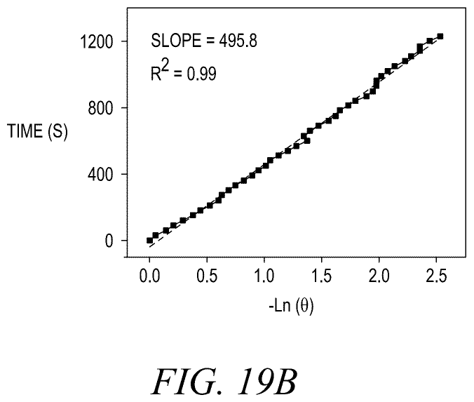

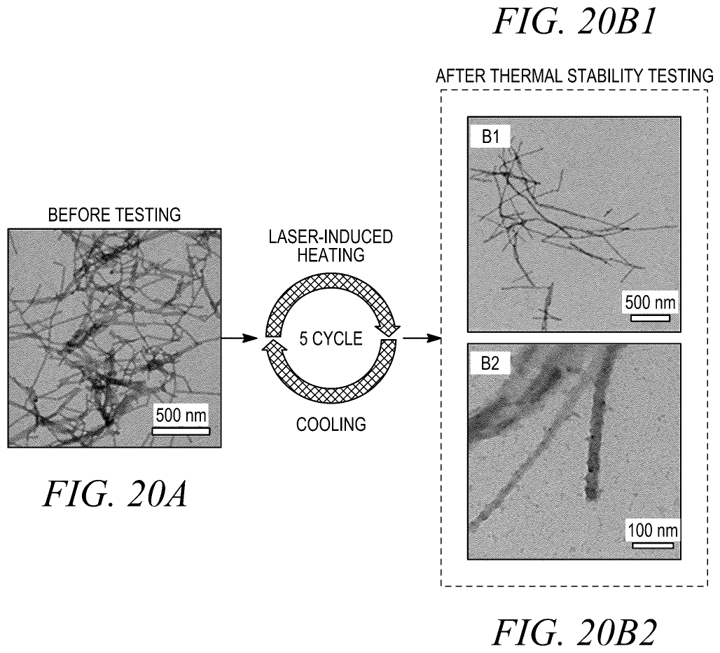

The heating profiles and maximum temperature were used to evaluate thermal performance, while the cooling profiles were used to calculate the photothermal conversion efficiency (r/). Calculation and example data can be found in the supporting information. First, when irradiated by an 808-nm laser, concentration-dependent (TMV@Fe³⁺-TA nanohybrids=0, 1.20, 2.40, 3.60, and 4.80 mg·mL⁻¹) temperature profiles show quicker temperature rise and higher maximum temperatures as the increase of concentrations. The temperature profile of pure TMV suspension (3.60 mg·mL⁻¹) was similar to that of DI water, while the maximum temperature for the TMV@Fe³⁺-TA suspension (63.6° C.) is significantly higher than the pure TMV's (3.3° C.). Further, power-dependent temperature profiles show the maximum temperature of TMV@Fe³⁺-TA nanohybrids (3.60 mg·mL⁻¹) increased from 37.0-82.8° C. as the laser power increased from 0.33-1.5 W·cm⁻² (FIG. 18). Finally, the η was averaged to be 33.2% based on three independent measurements under different power densities of 0.33, 0.50, and 1.00 W cm⁻², R²=0.99 (FIG. 19, Table 4). The value of η is close to these of pure Fe³⁺-TA and Fe³⁺-EGCG complexes, and higher than phenolic-based nanohybrids, such as Gd³⁺-DOTA-TMV@PDA and PVP@Fe³⁺-TA (summarized in Table 5). The thermal stability test showed that the photothermal performance of TMV@Fe³⁺-TA remained unchanged after five circles of irradiation. FIG. 4(c) shows the photothermal stability of TMV@Fe³⁺-TA (3.60 mg·mL⁻¹) with an 808-nm laser. The sample experienced five cycles of heating and cooling. Like the thermal stability test, the TEM images showed that supramolecular coatings had no obvious exfoliation after repeated irradiation (FIG. 20), suggesting that the TMV@Fe³⁺-TA nanohybrids are promising photothermal agents for PTT.

TABLE 4
Summary of maximum temperatures and efficiencies of
TMV@Fe³⁺-TA with different laser power

| Laser Power (W · cm⁻¹) | Maximum Temperature (° C.) | Efficiency (%) |
|---|---|---|
| 0.33 | 40.5 | 32.5 |
| 0.50 | 48.8 | 35.4 |
| 1.00 | 65.6 | 31.8 |

*Each sample was irradiated for 30 min to reach maximum temperature, and another 30 min to allow natural cooling.

TABLE 5
Summary of photothermal conversion efficiency in metal-
phenolic complexes and phenolic-based nanohybrids

| | Photothermal Conversion | |
|---|---|---|
| Materials | Efficiency η (%) | Ref |
| Fe³⁺-TA complex | 35.1 | 16 |
| Fe³⁺-EGCG complex | 30.3 | 16 |
| PVP@Fe³⁺-EA | 17.6 | 17 |
| Gd³⁺-DOTA-TMV@PDA | 28.9 | 3 |
| TMV@Fe³⁺-TA | 33.2 | This work |

Abbreviation: TA, tannic acid; EGCG, epigallocatechin gallate; PVP, Polyvinylpyrrolidone; EA, ellagic acid; PDA, polydopamine; Gd, Gadolinium; Fe, iron; TMV, tobacco mosaic virus; DOTA, monoamide-1,4,7,10-tetraazacyclododecane-N-′-N″-N″′tetraacetic acid.

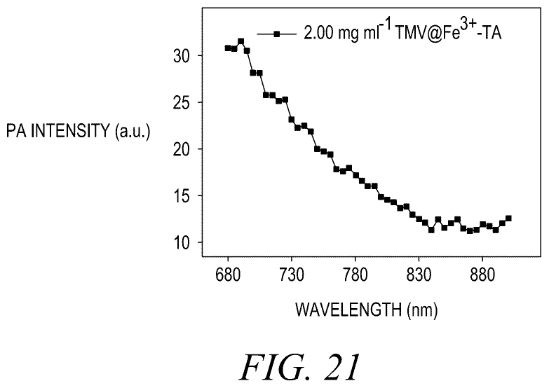

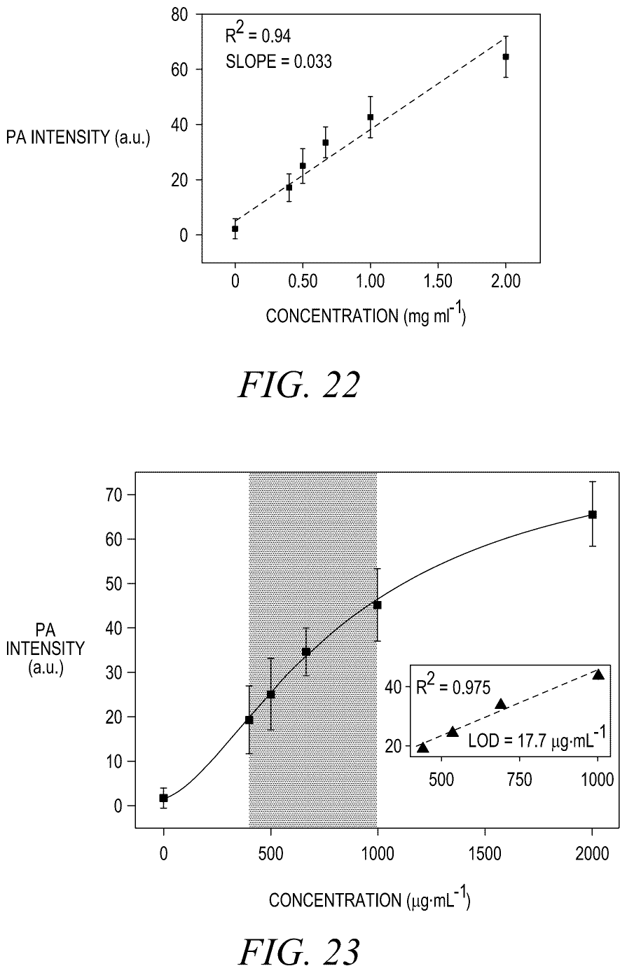

We further assessed the PA performance because the PA and photothermal properties are always correlated. TMV@Fe³⁺-TA (2.00 mg·mL⁻¹) exhibited clear PA signals from 680-900 nm (FIG. 21); the highest signal was at 680 nm. In contrast, native TMV at the same concentrations (2.00 mg·mL⁻¹) showed negligible PA signal enhancement (data not shown), indicating that the Fe³⁺-TA coating is responsible for the PA signals displayed by the TMV@Fe³⁺-TA. We selected the 680-nm laser as the light source and studied the concentration-dependent PA performance. Specifically, the PA signals of TMV@Fe³⁺-TA exhibited a good linear relationship (R²=0.94) from 0 to 2.00 mg·mL⁻¹. FIG. 4(d) shows the PA intensity of TMV@Fe³⁺-TA in elevated concentrations when illuminated by a 680-nm laser (n=9 regions of interest). The inset shows a dual PA-US image of the samples. The data was processed using ImageJ. (see also FIG. 22). Based on the previous method, the limit of detection (LOD) of PA was determined to be 17.7 μg·mL⁻¹ in DI water (FIG. 23). Moreover, we observed negligible PA intensity drop in the continuous irradiation of 680 nm laser for 15 min, which indicated their potential for prolonged PA imaging. FIG. 4(e) shows the time-dependent photostability test of TMV@Fe³⁺-TA (2.00 mg·mL⁻¹) for 15 min.

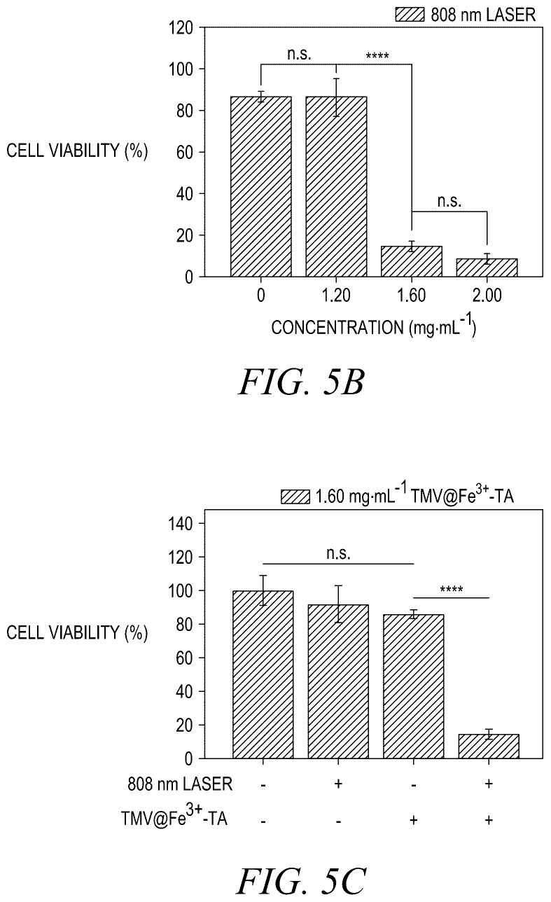

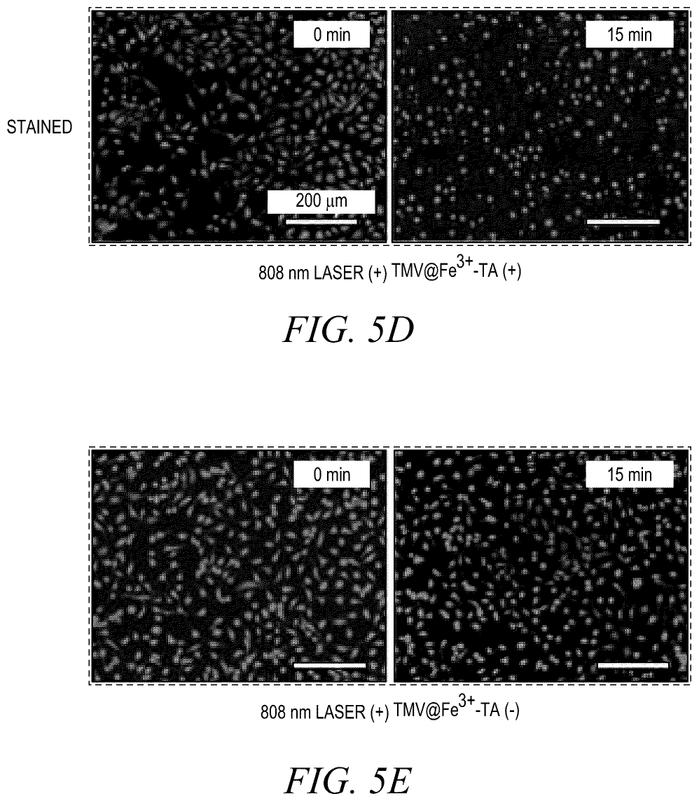

Finally, we evaluated the cytotoxicity of the TMV@Fe³⁺-TA and tested their photothermal cancer cell killing capabilities. The in vitro cell viability was conducted using human ovarian adenocarcinoma SKOV3 cells. After an incubation of 24 h, both TMV and TMV@Fe³⁺-TA of up to 2.00 mg·mL⁻¹ did not show obvious cytotoxicity. FIG. 5a shows the cell viability of SKOV3 cells determined by resazurin toxicology assay after incubating with different concentrations of TMV and TMV@Fe³⁺-TA for 24 h. Laser irradiation (808 nm laser, 1.0 W cm⁻² for 15 min) was applied on the SKOV3 cells incubated with different concentrations of TMV@Fe³⁺-TA (i.e., 0, 0.40, 0.80, 1.20, 1.60, and 2.00 mg·mL⁻¹, respectively). Cells were incubated in 1.60 mg·mL⁻¹ and treated by thermal bleaching (58° C., 15 min) as a positive control. FIG. 5b shows the concentration-dependent cell viability after photothermal treatment (808 nm laser, 1.0 W cm⁻² for 15 min) following 24 hours incubation with different concentrations of TMV@Fe³⁺-TA (0-2.0 mg·mL⁻¹). The cell viability decreased from 86% to 13% when the concentrations of TMV@Fe³⁺-TA increased from 1.20 mg·mL⁻¹ to 1.60 mg·mL⁻¹. Therefore, the concentration of 1.60 mg·mL⁻¹ was selected to evaluate the thermal ablation efficacy. FIG. 5c shows the comparative cell viability for different treatment regimens: 808 nm laser on (+) or off (−) for 15 min, combined with (+) or without (−) the incubation of 1.60 mg·mL⁻¹ TMV@Fe³⁺-TA for 24 h. (note that the experiments in FIGS. 5a-5c were conducted in triplicate). Compared to the cell viability of groups treated only with the laser (92%) or only with TMV@Fe³⁺-TA (85%), the cell viability of the group treated with both laser and TMV@Fe³⁺-TA decreased to 15%.

A fluorescence-based live/dead cell assay was further used to visualize the cytocompatibility and thermal ablation performance of TMV@Fe³⁺-TA at 1.60 mg·mL⁻¹ (FIGS. 5d, 5e and 24). Fluorescence images of SKOV3 are shown in FIG. 5(d) with a combination of laser irradiation (808 nm, 15 min) and TMV@Fe³⁺-TA (1.60 mg·mL⁻¹) incubation for 24 h and in FIG. 5(e) with laser irradiation only. (Staining: Calcein AM and PI. Significant difference: p<0.0001; and n.s.=no significant difference (p>0.05)). All cells were stained with Calcein-AM and propidium iodide (PI) dyes simultaneously to identify live cells (green fluorescence) and dead cells (red fluorescence). Fluorescent images demonstrate that significant cell death was caused by the combination of laser irradiation and TMV@Fe³⁺-TA incubation, which is consistent with data from cell viability assessment (FIG. 5c).

In summary, a one-step and versatile approach has been described for multifunctional coating on viral nanoparticles via metal-phenolic networks (MPNs). We demonstrated the miscellaneous combination of functional components (e.g., metal ions, dyes, and VNPs) to form coating that impart multifunction to viral nanoparticles, including combined imaging modalities (e.g., fluorescent labeling and photoacoustic imaging) and therapeutic effect (e.g., photothermal therapy). The cell-killing capacity was demonstrated to be effective. Since the obtained biohybrids were biocompatible and fully encapsulated, this strategy might also be used to protect virus-based vaccines from storage failure, especially in those developing regions where constant refrigeration is not always available. The multifunctional and biocompatible coating strategy will underpin a promising platform for proteinaceous therapeutics, virus-based recombinant vaccines, biosensing, catalyst, and more potential applications.

### Other Applications

Another application of the techniques described herein concerns the immuno-chemo-PTT combination therapy of the multifunctional viral nanoparticles described herein such as CisTMV@MPN. The combination immuno-chemo-PTT therapy induced by CisTMV@MPN refers to the use of multiple treatments simultaneously to enhance their therapeutic effects through synergistic effects. Combining chemotherapy and photothermal therapy with an immunomodulatory carrier agent can result in synergistic anti-tumor effects. Specifically, chemotherapy (e.g., cisplatin cargo) can induce cancer cell death; this is amplified by the PTT function of the MPN coating. Cancer cell death through multiple mechanisms will kill more tumor cells and release efficiently tumor associated antigens. The carrier (TMV in this example) is immunomodulatory as it presents danger signals and therefore recruits and activates immune cells to and within the tumor microenvironment. The recruited immune cells then process the TAAs released by chemo and PTT and therefore are synergistic. This combination therapy approach aims to increase the effectiveness of cancer treatment by targeting multiple pathways simultaneously, potentially leading to improved patient outcomes.

Another application of the techniques described herein concerns agricultural applications for the delivery of agrochemicals to load various active ingredients (AIs) as a functional component.

In general, the formation of the metal-phenolic network materials described herein involves 1) adding phenolics molecules (e.g., tannic acid, TA) to viral nanoparticles (e.g., TMV, TMGMV, CPMV, and PVX), the tannic acid molecules attach on the surface of TMV as a nomo-layer due to the hydrogen bonding and hydrophobic effect between TA and TMV. 2) then, by adding active ingredients (AIs) (e.g, crystal violet, oxytetracycline), miscellaneous AIs are able to bind with the free-floating TA molecules due to the π-π interactions between AIs and phenolic groups from TA molecules; 3) finally, by adding the metal ions (e.g., iron, zirconium, and gadolinium) as crosslinkers, the Al-bonded TA molecules are crosslinked and thicken the existed TA coating to form a supramolecular coating with AIs trapped; 4) purification.

For example, in some agricultural applications (e.g., Nematodes and anti-bacterial assay), the TMV@MPN*AI nanohybrids tend to attach to the surface of Nematodes (e.g. C. Elegant) or bacteria (e.g. E. Coli) due to its elongated shape. The drug is released to the microenvironment to achieve an effective treatment efficacy even with a lower drug amount.

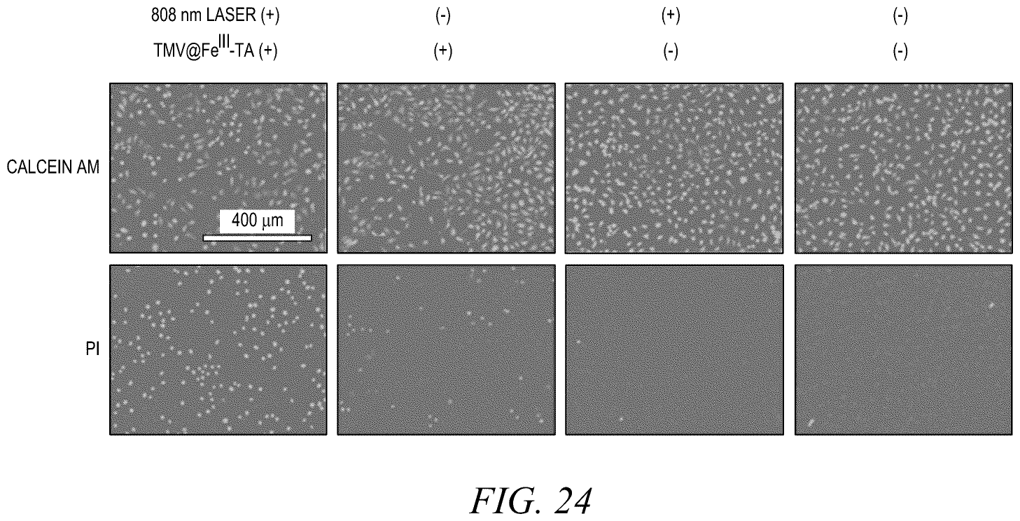

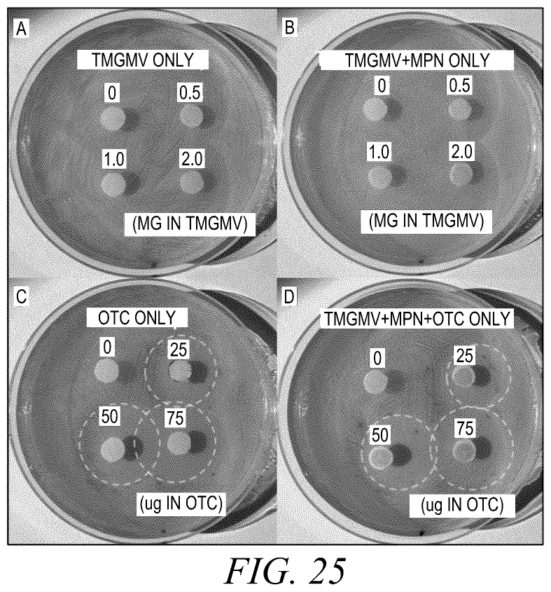

FIGS. 25a-25d show a disk diffusion assay for an anti-bacterial test. Different concentrations of a negative group (a) TMGMV only, (b) TMGMV only; of positive control group (c) OTC only, and experiment group (d) TMGMV+MPN+OTC are added to a filter disk, and then incubated on the surface of an agar substrate with bacterial before overnight incubation under 37° C. E. Coli is selected as a model bacterial. The dashed circle indicates the zone of inhibition. The diameters of zones are averaged after measurement three times. The experiments are performed in triplicate in different plates, and only the representative plates are shown.

### Fabrication and Characterization

The following discussion describes the fabrication and characterization techniques used in connection with the illustrative nanosystems described above. Those of ordinary skill in the art will recognize that the subject matter described herein is not limited to these particular systems or techniques, which are presented for illustrative purposes.

Regarding the materials used, tannic acid (TA), iron(III) chloride hexahydrate (FeCl₃·6H₂O, 97%), gadolinium (III) chloride hexahydrate (GdCl₃·6H₂O, 99%), zirconyl chloride octahydrate (ZrOCl₂·8H₂O, 98%), rhodamine 6g (Rh6G), Thiazole Orange (TO) were from Sigma-Aldrich. 2-amino-2-(hydroxymethyl)-1,3-propanediol (TRIS, >98%) was purchased from Tokyo Chemical Industry Co., Ltd. (TCI). Organic solvents including N,N-dimethylformamide (DMF, sequencing grade) and dimethyl sulfoxide (DMSO, certified ACS) were from Fisher Scientific International, Inc. (Hampton, NH). Propidium iodide (≥95%, PI) was purchased from Combi-Blocks (San Diego, CA, USA). Resazurin and McCoy's 5A medium were purchased from Sigma-Aldrich (Atlanta, GA, USA). TEM grids (200 mesh) were obtained from Ted Pella, Inc. High-purity water with a resistivity of 18.2 MΩ cm was obtained from an inline Millipore RiOs/Origin water purification system. All solutions were freshly prepared for immediate use in each experiment.

The plant viruses were propagated in and purified from the leaves of different plant species following established protocols. CPMV particles were extracted from Vigna unguiculata; PVX and TMV were isolated from the same plant species, Nicotiana benthamiana. The concentration of TMV was measured on a NanoDrop 2000 spectrophotometer (Thermo Fisher Scientific).

The integrity of plant virus nanoparticles (VNPs) was analyzed by size-exclusion fast protein liquid chromatography (FPLC) on a AKTA Explorer chromatography system equipped with a Superose6 column (GE Healthcare). Transmission electron microscopy (TEM) images were acquired using a JEOL JEM 1400 Plus operating at 80 kV with a Gatan 4k digital camera. Electron-dispersive X-ray spectroscopy (EDS) images were performed using a Thermo Fisher Talos 200X operating at 200 kV. Scanning TEM images and EDS mapping were performed by using Thermo Scientific software. Dynamic light scattering (DLS) measurements were performed on a Malvern NANO-ZS90 Zetasizer to acquire hydrodynamic sizes and zeta-potential values of the viral nanoparticles and biohybrids. UV-visible absorption measurements were performed on a BioTek Synergy H1 microplate reader. The ratio of A₂₆₀ to A₂₈₀ values was acquired using Thermo Scientific NanoDrop One^{c} Spectrophotometer. Temperature profiles and contours were recorded by FLIR CX-Series compact thermal imaging camera. Photoacoustic (PA) images and signals were collected using VisualSonics Vevo 2100-LAZR. PA signals were processed using ImageJ software.

Regarding the encapsulation of the plant VNPs with metal-phenolic networks, all solutions were freshly prepared for immediate use. The standard preparation process is described as follows: in a 2-mL tube, 100 μl of VNPs dispersion (2 mg·mL⁻¹) was first added dropwise to 660 μl of DI water with a vigorous vortex. Next, 20 μl of TA solution (4 mg·mL⁻¹) was added dropwise while gently vortexing followed by a one-time addition of 40 μl of the fresh metal solution (Fe³⁺ of 0.95 mg·mL⁻¹ as an example). The dispersion was then vigorously vortexed to yield the final concentrations (metal: 0.15 mM of FeCl₃·6H₂O, ZrOCl₂·8H₂O, and GdCl₃·6H₂O; TA: 0.050 mM; plant VNPs: 0.20 mg·mL⁻¹ of TMV, CPMV, and PVX). The pH was then raised to ˜8.0 by adding 80 μl of TRIS buffer (10 mM, pH 8.5). The resulting particles were then washed with DI water three times to remove excess metal-TA complexes, and the particles were pelleted by centrifugation (20000 g, 10 min). The supernatant was then completely removed, and pellets were redispersed in DI water to obtain VNPs@MPN biohybrid for further characterization. All syntheses were conducted under ambient atmosphere and room temperature.

A mixture of VNPs samples with the loading buffer (made of 62.5 mM Tris-HCl pH 6.8, 2% (w/v) SDS, 10% (v/v) glycerol, 0.01% (w/v) bromophenol blue, 10% (v/v) 2-mercaptoethanol) was boiled at 100° C. for 5 min. The resultant samples were loaded on 12% NuPAGE polyacrylamide gels run in 1×[3-(N-morpholino) propane sulfonic acid] buffer (MOPS, Invitrogen) for 40 min at 200 V. SeeBlue Plus2 pre-stained protein standards (Thermo Fisher Scientific) was used to provide the weight ladder markers. Following the run, the gels were stained with Coomassie Brilliant Blue and imaged on AlphaImager system (Protein Simple).

TA and fluorescent dyes were added into TMV dispersion with vigorous vortex followed by the addition of ZrOCl₂·8H₂O solution to yield the following final concentrations (dyes: 3.0 μM of Thiazole orange or Rhodamine 6G; ZrOCl₂·8H₂O: 0.15 mM; TMV: 0.20 mg·mL¹; TA: 0.050 mM). The pH was adjusted to ˜8.0 by TRIS buffer (80 mL, 10 mM, pH 8.5) and the system reacted for 30 min. The resulting particles were then washed with DI water three times to remove excess complex in the supernatant, the particles were centrifuged (20000 g, 10 min), the supernatant was completely removed, and the pellets were redispersed in DI water to obtain fluorescent TMV particles. The resulting particles were characterized by plate reader or fluorescent microscopy.

Regarding photoacoustic imaging of TMV@Fe³⁺-TA, a VisualSonics Vevo 2100 LAZR imaging system was used to take the PA signal. Samples were imaged using a 21 MHz-centered LZ 250 transducer and the peak energy is 45±5 mJ at 20 Hz at the source. The laser was calibrated and optimized before the sample measurement. The specimens were positioned at a depth of 1 cm from the transducer. The PA intensity spectra were obtained in the range of 680 to 900 nm. The PA and ultrasonic images of the samples were taken at 808 nm. All PA data were processed using Image J software. The average value and standard deviation of the PA intensity were calibrated based on the nine regions of interest per tube.

Regarding, cell culture and preparation, human ovarian adenocarcinoma (SKOV3) cells were cultured in McCoy's 5A Medium (1.5 mM L-glutamine, and 2.2 g·L⁻¹ sodium bicarbonate, supplemented with 10% fetal bovine serum and 1% penicillin-streptomycin). Cells are cultured under 5% CO₂ at 37° C. Cultures were given at least three passages before they were used for experiments. Cells were passaged to 75%˜80% confluency using 0.25% Trypsin-EDTA.

Regarding cell staining, the SKOV3 cells were seeded in a 24-well plate (50,000 cells per well) for 24 h, incubated with TMV@Fe³⁺-TA at a final concentration of 1.60 mg·mL⁻¹ for 24 h, were irradiated by 808 nm laser for 15 min and were incubated for 24 h. Afterward, 1000 μl of mixture solution of Calcein AM (2 M, live staining) and PI (6 M, dead staining) was added and incubated the cells for 30 min to stain the SKOV3 cells. After washing with PBS, the fluorescence images of each sample were captured by EVOS FL fluorescence microscope in GFP and RGP channels.

In Vitro photothermal therapy was performed at 808-nm irradiation. SKOV3 cells were divided in a 96-well plate at a concentration of 10,000 cells/well and were seeded for 24 h to allow the cells to attach to the plate. The medium was then replaced with a mixture of medium and TMV@Fe³⁺-TA dispersion at final concentrations of 1.60 mg·mL⁻¹ and then cultured for 24 hours. The experimental wells were exposed to an 808 nm laser with a power density of 1.0 W cm⁻¹ for 15 min. After incubation of 24 h, the wells were washed three times with phosphate-buffered saline (PBS) to remove the free TMV@Fe³⁺-TA particles. Resazurin dye solution equal to 10% of the culture medium volume was added to stain the cells, the cells were incubated for 3 h, and the cell viability was measured fluorometrically by monitoring the increase in fluorescence at an emission wavelength of 590 nm using an excitation wavelength of 560 nm. Complete medium without cells was used as blank for viability quantitative calculation. All cell experiments were conducted in triplicate.

Cells were prepared and stained using the same method as that described above for the concentrations of samples are 0, 0.20, 0.40, 0.80, 1.20, 1.60, and 2.00 mg·mL⁻¹. Cells with pure TMV particles at the same concentrations were used as control experiments. Complete medium without cells was used as blanks for viability quantitation.

The cell experiments described herein were conducted in triplicate. The average value and standard deviation of the PA intensity were calibrated based on the nine regions of interest per tube. All statistical analyses were processed using Origin 2018 or Image J software. All data were represented as the average ±standard deviation. Error bars represent the standard deviation of the means. Statistical analysis: a p-value less than 0.05 was statistically significant. (*p<0.05, **p<0.01, ***p<0.001, ****p<0.0001, n.s.=no significant difference (p>0.05).

The photothermal conversion efficiency (q) of the TMV@Fe³⁺-TA nanohybrids was determined according to known methods. The detailed calculation was given as follows.

$${\sum }_{i} {m}_{i} {C}_{p , i} \frac{d T}{dt} = {Q}_{N P} + {Q}_{s y s} - {Q}_{d i s s} \qquad ( 1 )$$

Where m and Cₚ are the mass (1.0 g) and specific heat capacity [4.18 J·(g·° C.)⁻¹] of the system components. Q_{NP} is the energy input of the TMV@Fe³⁺-TA nanoparticles, Qsys expresses the energy input of the container (i.e., quartz cuvette) with solvent (i.e., DI water), and the Q_{diss} represents the energy dissipation from the cuvette and solvent in the form of heat.

The term of energy input of TMV@Fe³⁺-TA nanoparticles, Q_{NP}, can be expressed as follow:

$${Q}_{N P} = I ( 1 - 1 {0}^{- {A}_{λ}} ) η \qquad ( 2 )$$

Where I is the laser power in Watt, A^{λ} is the absorbance of nanoparticles at the wavelength of the laser, and η is the photothermal conversion efficiency.

The term of energy dissipation from the system can be expressed below:

$${Q}_{d i s s} = h S ( T - {T}_{s u r r} ) \qquad ( 3 )$$

Where h is the heat transfer coefficient, S is the surface area of the container, and Tₛᵤᵣᵣ is the temperature of the surroundings.

When the temperature reaches an equilibrium (Tₘₐₓ), dT/dt=0. Therefore, eq 1 becomes

$$0 = {Q}_{N P} + {Q}_{s y s} - {Q}_{d i s s} \qquad ( 4 )$$

At this stage, the energy dissipation equals the total energy input terms, Q_{NP}+Q_{sys}:

$${Q}_{N P} + {Q}_{s y s} = {Q}_{d i s s} = h S ( {T}_{max} - {T}_{s u r r} ) \qquad ( 5 )$$

In the next experimental stage, the laser is turned off to allow energy dissipation only. So, the terms of Q_{NP} and Q_{sys} become zero, and eq 1 becomes

$${\sum }_{i} {m}_{i} {C}_{p , i} \frac{d T}{dt} = - {Q}_{d i s s} = - h S ( T - {T}_{s u r r} ) \qquad ( 6 )$$

Rearranging eq 6 gives

$$d t = - \frac{{\sum }_{i} {m}_{i} {C}_{p , i}}{hS} \frac{d T}{T - {T}_{s u r r}} \qquad ( 7 )$$

Integration of eq 7 gives

$$t = - \frac{{\sum }_{i} {m}_{i} {C}_{p , i}}{h S} ln \frac{T - {T}_{s u r r}}{{T}_{{max}^{- {T}_{s u r r}}}} \qquad ( 8 )$$

To determine the energy dissipation term, hS, the system time constant τₛ is introduced:

$${τ}_{s} = \frac{{\sum }_{i} {m}_{i} {C}_{p , i}}{h S} \qquad ( 9 )$$

And a dimensionless parameter theta is defined as

$$θ = \frac{T - {T}_{s u r r}}{{T}_{{max}^{- {T}_{s u r r}}}} \qquad ( 10 )$$

Substituting eqs 9 and 10 into eq 8 gives

$$t = - {τ}_{s} ln θ \qquad ( 11 )$$

By using eq 11, time constant τₛ can be determined by plotting linear regression of the t in second vs the negative natural logarithm of θ, plotted as FIG. 19. Recording starts (t=0) at the time when the laser is turned off and the temperature drops.

Q_{diss} can be determined independently by calculating hS′ from the cuvette and solvent in the absence of nanoparticles:

$${Q}_{d i s s} = {hS}^{′} ( {T}_{max , {H}_{2} O} - {T}_{s u r r} ) \qquad ( 12 )$$

The photothermal conversion efficiency can be determined by the equation:

$$η = \frac{h S ( {T}_{max} - {T}_{s u r r} ) - {Q}_{d i s s}}{I ( 1 - {10}^{- {A}_{λ}} )} \qquad ( 13 )$$

The calculation of limit of detection (LOD) of the TMV@Fe³⁺-TA nanohybrids was determined according to the previous method. The detailed calculation was given as follows. The LOD was determined using the limit of blank (LOB). The LOB is defined as the highest signal generated from a sample that contains no analyte. It is calculated by taking replicate of a blank sample and finding the mean and standard deviation (SD).

$$LOB = {mean}_{blan k} + 1.645 ( S {D}_{b l a n k} ) \qquad ( 14 )$$

The LOB encompasses 95% of observed blank values while the remaining 5% contains a response that could have been generated from a low analyte concentration. The LOD is defined as the minimum analyte concentration that can be reliably distinguished from the LOB.

$$L O D = LOB + 1.645 ( S {D}_{low concentration} ) \qquad ( 15 )$$

The LOD represents an analyte concentration in which 95% of measured samples are distinguishable from the LOB while the remaining 5% erroneously appear to contain no analyte. In our experiments, the LOB and LOD were calculated from FIG. 23 using values of PA intensity when the concentrations of nanohybrids are 0 and 400 μg·mL⁻¹, respectively.

### Additional Figures

FIGS. 6a-6d show representative molecular structures. In particular, FIG. 6a show Tannic acid (TA), FIG. 6b shows the coordination complex of Mⁿ⁺-TA. R represents the remainder of the TA molecules, and Mⁿ⁺ represents the central metal ions with the oxidation number of n. FIG. 6c shows the molecular structure of Rhodamine 6G (Rh6G) and FIG. 6d shows the molecular structure of Thiazole Orange (TO).

FIGS. 7(a)(1)-7(a)(3) and 7(b)(1)-7(b)(3) show characterization data of the prepared VNPs. The UV absorption spectra of CPMV (FIG. 7a1), PVX, (FIG. 7a2) and TMV (FIG. 7a3) nanoparticles are shown, where the dashed lines indicate the absorption wavelengths due to the capsid protein (260 nm) and viral RNA (280 nm). FPLC chromatograms of CPMV (FIG. 7b1), PVX (FIG. 7b2), and TMV (FIG. 7b3) nanoparticles are shown, where particles were eluted using potassium phosphate buffer (0.01 μM for TMV and 0.1 μM for CPMV and PVX) at the flow rate of 0.5 mL min⁻¹. Co-elution of the viral constructs indicate the presence of intact particles: The dashed curve represents viral capsid proteins, and the solid curve represents RNA detected at 260 nm and 280 nm, respectively.

FIGS. 8a-8e show the optimization of the morphology of TMV@Fe³⁺-TA. TEM images showing the Fe³⁺-TA coating morphology on TMV in final solutions with different concentrations. The ratios of the concentration of iron ([Fe³⁺]) and tannic acid ([TA]) in final solution were listed as follow. [Fe³⁺]: [TA]=0:0 (FIG. 8a), 0.020:0.0070 (FIG. 8b), 0.080:0.027 (FIG. 8c), 0.15:0.050 (FIG. 8d), and 0.30:0.10 (FIG. 8e). All values were in μM. The concentration of TMV used in all synthesis was kept at 0.20 mg·mL⁻¹. Shared scale bars were 100 nm in FIGS. 8a-8e. The formulation [Fe³¹]: [TA]=0.15:0.050 was selected for further experiments and was marked in the dashed box. Note that none of the samples were negative stained.

FIGS. 9a-9b depict results of serial dilution proving dispersity of TMV@Fe³⁺-TA. The dilution of original stock was set to 1:3, 1:6, 1:10, 1:100 in water-based buffer. The good linearity (R²=0.953 and 0.991) indicates good dispersity of TMV@Fe³⁺-TA at concentration up to 2.0 mg·mL⁻¹. The absorbance of TMV@Fe³⁺-TA (FIG. 9a) and TMV (FIG. 9b) were determined at 808 and 260 nm, respectively. The highest concentrations of two samples marked by asterisks are determined to be 2.00 mg·mL⁻¹.

FIGS. 10a-10b illustrate the Fe³⁺-TA coating on TMV by EDS linear scan. FIG. 10a shows the merged representative EDX image of TMV@Fe³⁺-TA nanohybrids. The EDS linear scan of nanohybrid was indicated in the dashed line. FIG. 10b shows that the signals of nitrogen, carbon, oxygen, and iron are in the corresponding dashed line. The existence of the Fe signal indicated the successful coating of the Fe³⁺-TA coordination complex on TMV.

FIGS. 11a-11b show UV-Vis spectra of Gd³⁺-TA and Zr⁴⁺-TA coatings on TMV. FIG. 11a shows TMV@Gd³⁺-TA nanohybrids and FIG. 11b shows TMV@Zr⁴⁺-TA nanohybrids with varying final concentrations of precursors; scans from 280 to 900 nm. The molar ratio of Gd³⁺ to TA was adjusted to 3:1. The molar ratio of metal ions to TA=0.30:0.10, 0.15:0.050, 0.080:0.024, and 0:0. [Zr⁴⁺][TA]=0.24:0.078, 0.16:0.052, 0.07:0.026, and 0:0 (from top to down). All values are in μM. Insets: magnification revealed minor relative differences.

FIG. 12 shows the size distribution of metal-dependent TMV@MPN nanohybrids. Dynamic light scattering data shows the sizes of TMV@Fe³⁺-TA, TMV@Gd³⁺-TA, and TMV@Zr⁴⁺-TA in number.

FIG. 13 shows photographs of various MPN coatings on TMV nanoparticles. Photographs of pure TMV, and the optimized TMV@Fe³⁺-TA, TMV@Zr⁴⁺-TA, and TMV@Gd³⁺-TA suspensions at 1.0 mg·mL⁻¹ are shown.

FIGS. 14a-14b shows data estimating the fluorescence performance of TMV@Zr⁴⁺-TA-Rh6G nanohybrids. FIG. 14a shows the concentration-dependent fluorescence intensity of Rh6G dye in DI water (excitation/emission=488 nm/555 nm). All experiments were performed in triplicate. FIG. 14b estimates the fluorescence performance of TMV@Zr⁴⁺-TA-Rh6G nanohybrids. The linear regression (R²=0.995) is based on the 0-7.39 μM Rh6G solution. By collating the fluorescence intensity (1.02×10⁴) of TMV@Zr⁴⁺-TA-Rh6G with standard fluorescent curve (a), the fluorescence intensity of such nanohybrids is roughly equivalent of 0.49 μM free Rh6G dye in DI water. It is notable that the dye is 3.0 M in synthesizing such TMV@Zr⁴⁺-TA-Rh6G nanohybrids. The loss of fluorescence intensity can be explained by the quenching effect of TA molecules.

FIGS. 15a-15c show the size distribution of various VNPs before and after Fe³⁺-TA coating. The formation of Fe³⁺-TA shifted all groups of VNPs to larger sizes: TMV (FIG. 15a); CPMV (FIG. 15b); PVX (FIG. 15c). Aggregation was apparent for the CPMV formulation.

FIGS. 16a1-16a2, 16b1-16b2 and 16c1-16c3 show magnified TEM images to show Fe³⁺-TA coating on VNPs. Compared to pristine (FIG. 16a1) PVX, (FIG. 16b1) CPMV, and (FIG. 16c1) TMV, the morphological changes of (FIG. 16a2) PVX@Fe³⁺-TA, (FIG. 16b2) CPMV@Fe³⁺-TA, and (FIG. 16c3) TMV@Fe³⁺-TA showed successful coating. Note that none of the samples were negative stained. Sample preparation and TEM equipment were same. The differences of contrast between the blur images of uncoated VNPs (a1-c1) and distinct images of coated VNPs (a2-c2) also indicated successful coatingsb

FIGS. 17a-17c show an SDS-PAGE analysis of the viral coat proteins (CPs) from VNPs@Fe³⁺-TA vs. wild type VNPs. FIG. 17a compares CPMV@Fe³⁺-TA vs. native CPMV; FIG. 17b compares PVX@Fe³⁺-TA vs. native PVX; and FIG. 17c compares TMV@Fe³⁺-TA vs. native TMV. CPs from both coated and uncoated VNPs displayed identical electrophoretic profiles, which indicates that their chemical composition remained unchanged after coating. The small and large capsid protein units of CPMV were detected at 24 and 41 kDa, respectively in the lanes from both coated and uncoated CPMV samples. The capsid proteins of PVX and TMV were also detected at expected weights (28 and 18 kDa, respectively), regardless of the presence of coating. PVX@Fe³⁺-TA showed similar additional band as TMV@Fe³⁺-TA corresponding to the viral protein dimers. The additional bands at ˜49 kDa in FIG. 17b and at ˜39 kDa in FIG. 17c correspond to dimers, which possibly can be explained by the intertwining effect of Fe³⁺-TA complex.

FIG. 18 shows power-dependent temperature profiles of TMV@Fe³⁺-TA. Temperature profiles of TMV@Fe³⁺-TA suspensions (3.60 mg·mL⁻¹) irradiated by 808 nm laser (1.5, 1.0, 0.50, 0.33 W cm⁻²) for 10 min are shown. The experiments were conducted in a plastic cuvette that has a larger hS parameter for magnification of temperature differences.

FIGS. 19a-19b show data for calculating the photothermal conversion efficiency. FIG. 19a shows the representative temperature profiles of TMV@Fe³⁺-TA (3.60 mg·mL⁻¹), pure TMV (3.60 mg·mL⁻¹), and DI water with heating-up duration of 30 min and cooling-down duration of 30 min at 808-nm laser (power 1.00 W·cm⁻²). FIG. 19b shows the linear time date vs. −Ln (θ) obtained from the cooling period of FIG. 19a. The system time constant τₛ equals the value of slope (495.8). The calculation method was described above, specifically, equations (12,13).

FIGS. 20a and 20b1-20b2 show a photothermal stability assessment by TEM. A TEM image of TMV@Fe³⁺-TA nanohybrid is shown before (FIG. 20a) and after (FIG. 20b1) the photothermal test with a 808 nm laser. The magnification in (FIG. 20b2) showed no obvious exfoliation of the coating after five cycles of photothermal heating and cooling, indicating that the nanoparticles can function well in normal photothermal therapy (maximum temperature: ˜63° C., 10 min for each heating-up and cooling-down period, 100 min in total).

FIG. 21 shows the photoacoustic spectrum of TMV@Fe³⁺-TA. The suspension (2.00 mg·mL⁻¹) was scanned over a wavelength range of 680-900 nm. The PA intensity decreases as the wavelength increased. 680 nm was determined as the optimal laser wavelength for TMV@Fe³⁺-TA.

FIG. 22 shows a linear analysis of TMV@Fe³⁺-TA at different concentrations. TMV@Fe³⁺-TA nanohybrids at different concentrations were measured by a 680 nm pulse laser in the range of 0-2.00 mg·mL⁻¹, which showed a good linear relationship.

FIG. 23 shows the limit of detection (LOD) of PA. PA intensity LODs for TMV@Fe³⁺-TA nanohybrids is 17.7 μg·mL⁻¹. The calculation was based on linear range of 400-1000 μg·mL⁻¹ (R²=0.975). Measurements were performed in triplicate, error bars=standard deviation. The calculation method was described above, specifically, equation (14-15).

FIG. 24 shows calcein AM and PI-stained SKOV3 cells. Different treatments on SKOV3 were labeled with laser irradiation (808 nm, 15 min) on (+) or off (−), and with the incubation (+) of 1.60 mg·mL⁻¹ TMV@Fe³⁺-TA for 24 h or with incubation of buffer (−). Shared scale bars are 400 μm. The merged images in FIG. 4d were processed by ImageJ.

The particular structures and methods described above have been presented for illustrative purposes and not as a limitation on the subject matter described herein. More generally, in one aspect, a method is presented for functionalizing plant viral nanoparticles (VNPs). In accordance with the method, a plant VNP is selected. A metal ion and a phenolic compound that form a metal-phenolic network (MPN), and at least one functional component that adheres to the MPN are also selected. A nanohybrid structure is synthesized from a solution of the selected metal, the selected phenolic compound and the selected functional component such that the synthesized nanohybrid structure has an MPN coating encapsulating the plant VNP with the functional component being embedded in the MPN coating.

In some embodiments the plant VNP is selected from the group consisting of a tobacco mosaic virus (TMV), a cowpea mosaic virus (CPMV) and a potato virus X (PVX).

In some embodiments 3 the metal ion is selected from the group consisting of FE³⁺, Zr⁴⁺ and Gd³⁺.

In some embodiments the phenolic compound is selected from the group consisting of tannic acid (TA), epigallocatechin gallate (EGCG), ellagic acid (EA) and polydopamine (PDA).

In some embodiments the functional component includes a fluorophore.

In some embodiments the functional component includes at least one therapeutic active ingredient.

In some embodiments the therapeutic active ingredient is selected from the group consisting of a medical drug, a pesticide, a bactericide and a fungicide.

In some embodiments the medical drug is cisplatin.

In some embodiments the at least one fluorescent die is selected from the group consisting of rhodamine 6G and thiazole orange.

In some embodiments the at least one functional component is selected such that the synthesized nanohybrid structure is functionalized for performing a theranostic function.

In some embodiments the at least one functional component is selected such that the synthesized nanohybrid structure is functionalized for performing photoacoustic imaging (PAI).

In some embodiments the at least one functional component is selected such that the synthesized nanohybrid structure is functionalized for performing photothermal therapy.

In some embodiments the at least one functional component is selected such that the synthesized nanohybrid structure is functionalized for performing chemotherapy.

In some embodiments the at least one functional component includes functional components for performing photothermal therapy and chemotherapy.

In some embodiments the at least one functional component is selected such that the synthesized nanohybrid structure is functionalized for performing fluorescent labeling.

In another aspect, a method is presented of imaging functionalized viral nanoparticles. In accordance with the method, synthesized nanohybrid particles are irradiated with light. The synthesized nanohybrid particles each have an MPN coating encapsulating a plant VNP that has been functionalized to provide a photoacoustic signal. A photoacoustic signal is received from the synthesized nanohybrid particles.

In some embodiments the method further includes administering the synthesized nanohybrid particles to a subject and identifying a location of the synthesized nanohybrid particles within the subject.

In some embodiments the method further includes determining a concentration of the synthesized nanohybrid particles within the subject.

In yet another aspect, a method of treating cancerous tissue is presented. In accordance with the method, synthesized nanohybrid particles are administered to a subject. The synthesized nanohybrid particles each have an MPN coating encapsulating a plant VNP that has been functionalized to provide photothermal therapy. A treatment site is irradiated at which the cancerous tissue is located to heat and kill the cancerous tissue.

In some embodiments administering the synthesized nanohybrid particles includes intravenously or intratumor injecting the synthesized nanohybrid particles to the subject.

In some embodiments the plant VNP acts as an immunomodulatory agent to reverse immune suppression, either conferred by the immunomodulatory properties of the plant VNP or inferred through an immunomodulatory cargo.

In some embodiments the synthesized nanohybrid particles are also functionalized to provide chemotherapy.

In some embodiments the synthesized nanohybrid particles have a medical drug embedded in the MPN coating for performing the chemotherapy.

In some embodiments the medical drug is cisplatin.

In some embodiments the plant VNP is TMV and the MPN is Fe³⁺-TA. The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein but may be modified within the scope and equivalent of the appended claims.

1. A method of functionalizing plant viral nanoparticles (VNPs), comprising:
selecting a plant VNP;
selecting a metal ion and a phenolic compound that form a metal-phenolic network (MPN) and at least one functional component that adheres to the MPN; and
synthesizing a nanohybrid structure from a solution of the selected metal, the selected phenolic compound and the selected functional component such that the synthesized nanohybrid structure has an MPN coating encapsulating the plant VNP with the functional component being embedded in the MPN coating.

2. The method of claim 1 wherein the plant VNP is selected from the group consisting of a tobacco mosaic virus (TMV), a cowpea mosaic virus (CPMV) and a potato virus X (PVX).

3. The method of claim 1 wherein the metal ion is selected from the group consisting of FE³⁺, Zr⁴⁺ and Gd³⁺.

4. The method of claim 1 wherein the phenolic compound is selected from the group consisting of tannic acid (TA), epigallocatechin gallate (EGCG), ellagic acid (EA) and polydopamine (PDA).

5. The method of claim 1 wherein the functional component includes a fluorophore.

6. The method of claim 1 wherein the functional component includes at least one therapeutic active ingredient.

7. The method of claim 6 wherein the therapeutic active ingredient is selected from the group consisting of a medical drug, a pesticide, a bactericide and a fungicide.

8. The method of claim 6 wherein the medical drug is cisplatin.

9. The method of claim 1 wherein the at least one fluorescent die is selected from the group consisting of rhodamine 6G and thiazole orange.

10. The method of claim 1 wherein the at least one functional component is selected such that the synthesized nanohybrid structure is functionalized for performing a theranostic function.

11. The method of claim 1 wherein the at least one functional component is selected such that the synthesized nanohybrid structure is functionalized for performing photoacoustic imaging (PAI).

12. The method of claim 1 wherein the at least one functional component is selected such that the synthesized nanohybrid structure is functionalized for performing photothermal therapy.

13. The method of claim 1 wherein the at least one functional component is selected such that the synthesized nanohybrid structure is functionalized for performing chemotherapy.

14. The method of claim 1 wherein the at least one functional component includes functional components for performing photothermal therapy and chemotherapy.

15. The method of claim 1 wherein the at least one functional component is selected such that the synthesized nanohybrid structure is functionalized for performing fluorescent labeling.

16. A method of imaging functionalized viral nanoparticles, comprising:
irradiating with light synthesized nanohybrid particles each having an MPN coating encapsulating a plant VNP that has been functionalized to provide a photoacoustic signal; and
receiving a photoacoustic signal from the synthesized nanohybrid particles.

17. The method of claim 16 further comprising administering the synthesized nanohybrid particles to a subject and identifying a location of the synthesized nanohybrid particles within the subject.

18. The method of claim 17 further comprising determining a concentration of the synthesized nanohybrid particles within the subject.

19. A method of treating cancerous tissue, comprising:
administering to a subject synthesized nanohybrid particles each having an MPN coating encapsulating a plant VNP that has been functionalized to provide photothermal therapy; and
irradiating a treatment site at which the cancerous tissue is located to heat and kill the cancerous tissue.

20. The method of claim 19 wherein administering the synthesized nanohybrid particles includes intravenously or intratumor injecting the synthesized nanohybrid particles to the subject.

21. The method of claim 19, wherein the plant VNP acts as an immunomodulatory agent to reverse immune suppression, either conferred by the immunomodulatory properties of the plant VNP or inferred through an immunomodulatory cargo.

22. The method of claim 19 wherein the synthesized nanohybrid particles are also functionalized to provide chemotherapy.

23. The method of claim 22 wherein the synthesized nanohybrid particles have a medical drug embedded in the MPN coating for performing the chemotherapy.

24. The method of claim 23 wherein the medical drug is cisplatin.

25. The method of 24 wherein the plant VNP is TMV and the MPN is Fe³⁺-TA.
